(19) United States
(12) Patent Application Publication
(10) Pub. No.: US 2025/0195848 A1
(43) Pub. Date: Jun. 19, 2025

(54) INFLATABLE BALLOONS WITH FLOW CHANNELS

(71) Applicant: EDWARDS LIFESCIENCES CORPORATION, Irvine, CA (US)

(72) Inventor: David Maimon, Atlit (IL)

(73) Assignee: EDWARDS LIFESCIENCES CORPORATION, Irvine, CA (US)

(21) Appl. No.: 19/033,695

(22) Filed: Jan. 22, 2025

Related U.S. Application Data
(63) Continuation of application No. PCT/US2023/028610, filed on Jul. 25, 2023.
(60) Provisional application No. 63/394,242, filed on Aug. 1, 2022.

(51) Int. Cl. A61M 25/10; A61F 2/24

(52) U.S. Cl. CPC A61M 25/1002; A61F 2/2433; A61M 25/10184; A61M 2205/332; A61M 2205/3334

(57) ABSTRACT

The present disclosure relates to inflatable balloons designed to permit blood flow through channels formed along and optionally around their outer surfaces. Such a balloon can include least two lobes spiraling around the balloon's axis and along the length of the central portion, and at least two depressed portions extending between the helical lobes. When the balloon is inflated within a target lumen, helical channels are provided between the depressed portions and an inner surface of the target lumen to allow blood to flow through the channels and across the balloon. The balloon can be inflated in a gradual manner, optionally based at least in part on measurements from one or more sensor(s) that can be attached to the balloon, such as flow sensors configured to measure flow through the helical channels, and/or force sensors configured to measure force applied against the balloon during inflation thereof.

### CROSS-REFERENCE TO RELATED APPLICATIONS

This application is a continuation of PCT Application No. PCT/US2023/028610, filed Jul. 25, 2023, which claims the benefit of U.S. Provisional Application No. 63/394,242, filed Aug. 1, 2022, each of which is incorporated by reference herein in its entirety.

### FIELD

The present disclosure relates to apparatuses and methods that can be used in the treatment of heart valve disease, including balloon valvuloplasty and the delivery of transcatheter heart valves.

### BACKGROUND

Heart valve disease is a serious problem that involves the malfunction of one or more valves of the heart. The malfunction can manifest itself in a variety of manners. For example, valve stenosis is the calcification or narrowing of a native heart valve. As a result, the native heart valve is not able to completely open and blood flow through the native valve is impeded or restricted. Another example of heart valve disease is valve insufficiency. Valve insufficiency is the failure of a native heart valve to close properly to prevent leaking, or backflow, of blood through the valve.

Various methods have been developed to treat heart valve disease. Some of these methods require a balloon member that is expanded within the native heart valve. For example, a balloon member can be used in a valvuloplasty procedure where the balloon member is positioned within the native heart valve and expanded to increase the opening size (i.e., flow area) of the native heart valve and thereby improve blood flow. Another procedure that can be performed is a valve replacement, in which a native heart valve is replaced by an artificial heart valve. The implantation of an artificial heart valve in the heart can also involve the expansion of a balloon member in the valve annulus. For example, the balloon member can be used to increase the size of the native valve prior to implantation of the artificial valve and/or it can be used to expand and deploy the artificial valve itself.

### SUMMARY

The expansion of a balloon member within a native valve or other vascular passageway can temporarily block or restrict blood flow through the passageway. If blood flow is blocked or restricted in the passageway for too long, serious injury or death can occur. Furthermore, in the case of valve replacement, the positioning of the artificial heart valve may be complicated by the buildup of pressure in the left ventricle. Accordingly, valvuloplasty and valve replacement procedures, and other similar procedures which utilize expandable balloon members, must generally be performed quickly and/or with a heart pacing procedure, so that the balloon member is inflated for only a brief period. The present disclosure is directed toward inflatable balloons that permit perfusion of blood around the balloon while a balloon is inflated in a passageway such as a patient lumen or a prosthetic valve.

According to one aspect of the disclosure, a delivery apparatus comprises a balloon movable between a deflated state and an inflated state. The balloon comprises an internal cavity and a central portions disposed around the cavity. The central portion comprises at least two lobes spiraling around a longitudinal axis of the balloon.

In some aspects, the central portions extends along a length between a leading portion and a trailing portion of the balloon.

In some aspects, the at least two lobes extend along the length of the central portion.

In some aspects, the central portion further comprises at least two depressed portions extending between the lobes.

In some aspects, the delivery apparatus further comprises a balloon catheter coupled to the balloon.

In some aspects, the balloon catheter comprises a balloon catheter lumen in fluid communication with the cavity of the balloon via one or more openings of the balloon catheter.

In some aspects, when the balloon is in the inflated state within a target lumen, at least two channels are provided between the depressed portions and an inner surface of the target lumen to allow blood to flow through the channels and across the balloon

In some aspects, the lobes and the depressed portions together form a cross-sectional contour devoid of acute or right angles between the lobes and the depressed portions.

In some aspects, the balloon is not wound around any other shaft or catheter extending along the longitudinal axis.

In some aspects, the delivery apparatus is devoid of constriction elements helically disposed around the balloon.

In some aspects, the delivery apparatus is devoid of biasing means disposed in the cavity, configured to press against the balloon in the inflated state to force it into a helically-shaped configuration.

In some aspects, the at least two lobes comprise three lobes, and wherein the at least two depressed portions comprise three depressed portions.

In some aspects, the leading and the trailing portions taper, in the inflated state, from the respective ends of the central portion to narrower diameters at their opposite ends.

In some aspects, the leading and the trailing portions have non-circular cross-sectional shapes along at least a portion of tapering segments thereof in the inflated state.

In some aspects, the delivery apparatus further comprises at least one flow sensor configured to detect the presence of blood flow across the balloon.

In some aspects, the at least one flow sensor is attached to the balloon.

In some aspects, the at least one flow sensor is attached to at least one of the depressed portions.

In some aspects, the delivery apparatus further comprises at least one force sensor.

In some aspects, the at least one force sensor is attached to a lobe outer surface of at least one of the lobes.

In some aspects, the lobe outer surface defines a width that is at least as great as the width of the force sensor.

According to one aspect of the disclosure, a method for inflating a balloon comprises delivering the balloon in a deflated state thereof through the vasculature of a patient to the treatment site, injecting inflation fluid into the balloon, and receiving information regarding blood flow across the balloon.

In some aspects, the balloon is mounted on a balloon catheter.

In some aspects, the balloon comprises an internal cavity and a central portion disposed around the cavity.

In some aspects, injecting inflation fluid into the balloon comprises injecting inflation fluid into the cavity of the balloon.

In some aspects, the method further comprises, based at least in part on the received information, stopping the injection of the inflation fluid into the balloon.

In some aspects, injection of the inflation fluid into the internal cavity of the balloon inflates the balloon within a target lumen such that the central portion comprises at least two lobes spiraling around a longitudinal axis of the balloon and along a length of the central portion, and at least two depressed portions extending between the lobes.

In some aspects, injecting the inflation fluid comprises injecting a first volume of the inflation fluid into the balloon, and injecting a second volume of the inflation fluid into the balloon, wherein the second volume of the inflation fluid is determined based at least in part on the received information.

In some aspects, the first volume of the inflation fluid is a first predetermined fraction of a nominal inflation volume.

In some aspects, the method further comprises injecting an additional volume of the inflation fluid into the internal cavity of the balloon until a first predetermined condition is satisfied.

In some aspects, the method further comprises measuring an amount of force between the balloon and tissue of the patient, or between the balloon and a prosthetic device, wherein the first predetermined condition is associated with the measured amount of force.

In some aspects, the method further comprises measuring the velocity of blood flow across the balloon, the received information regarding blood flow across the balloon based at least in part on the measured velocity.

In some aspects, the method further comprises, based at least in part on the measured velocity of blood flow, determining a desired inflation rate, the desired inflation rate negatively correlated with the measured velocity of blood flow, wherein the received information regarding blood flow across the balloon is based at least in part on the desired inflation rate.

In some aspects, the rate of inflation of the balloon is based at least in part on the determined desired inflation rate.

In some aspects, the method further comprises comparing the measured velocity of blood flow to a plurality of respective predetermined parameters, the desired inflation rate determined based at least in part on an outcome of the comparison to the respective predetermined parameters.

In some aspects, the method further comprises, based at least in part on the measured velocity of blood flow, determining a desired inflation time, the desired inflation time period positively correlated with the measured velocity of blood flow, wherein the received information regarding blood flow across the balloon is based at least in part on the determined desired inflation time.

According to some aspects of the disclosure, there is provided a delivery apparatus comprising a balloon movable between a deflated state and an inflated state, and a balloon catheter coupled to the balloon. The balloon comprises an internal cavity, and a central portion disposed around the cavity and extending along a length between a leading portion and a trailing portion of the balloon. The central portion comprises at least one helical lobe extending along the length of the central portion, and at least one depressed portions extending adjacent the at least one lobes. The balloon catheter comprises a balloon catheter lumen in fluid communication with the cavity of the balloon via one or more openings of the balloon catheter. When the balloon is in the inflated state within a target lumen, at least one channel is provided between the at least one depressed portion and an inner surface of the target lumen to allow blood to flow through the channel and across the balloon.

In some examples, the at least one lobe comprises at least two lobes, the at least one depressed portion comprises at least two depressed portions extending between the at least two lobes, and the at least one channel comprises at least two channels.

In some examples, the at least one lobe is a helical lobe spiraling around a longitudinal axis of the balloon, and the at least one channel is a helical channel.

In some examples, the delivery apparatus further comprises at least one sensor.

In some examples, the at least one sensor is a force sensor or a pressure sensor.

In some examples, the at least one sensor can comprise a flow sensor.

In some examples, the at least one flow sensor is configured to detect the presence of blood flow across the balloon.

According to some aspects of the disclosure, a method of deploying a balloon is provided. The method comprises delivering a balloon in a deflated state thereof, mounted on a balloon catheter, through the vasculature of a patient to a treatment site, the balloon comprising an internal cavity and a central portion disposed around the cavity. The method further comprises inflating the balloon within a target lumen in a manner that forms at least one channel through which blood is permitted to flow across the balloon.

According to some aspects of the disclosure, a method is provided. The method comprises delivering a balloon in a deflated state thereof, mounted on a balloon catheter, through the vasculature of a patient to a treatment site, the balloon comprising an internal cavity and a central portion disposed around the cavity. The method further comprises injecting inflation fluid into the internal cavity of the balloon. The method further comprises receiving information regarding the presence of blood flow across the balloon. The injection of the inflation fluid into the internal cavity of the balloon inflates the balloon within a target lumen, wherein blood is permitted to flow through at least one channel formed between the balloon and an inner surface of the target lumen.

In some examples, the method further comprises, based at least in part on the received information, stopping the injection of the inflation fluid into the balloon.

In some examples, the injection of the inflation fluid comprises injecting a first volume of the inflation fluid into the balloon and injecting a second volume of the inflation fluid into the balloon.

In some examples, the second volume of the inflation fluid is determined based at least in part on the received information.

In some implementations, the first volume of the inflation fluid is a first predetermined fraction of a nominal inflation volume.

In some examples, the method further comprises injecting an addition volume of the inflation fluid into the internal cavity of the balloon until a first predetermined condition is satisfied.

In some examples, the method comprises injecting an additional volume of the inflation fluid into the internal cavity of the balloon until a second predetermined condition is satisfied.

In some examples, the method further comprises measuring the velocity of blood flow across the balloon, the received information regarding the blood flow across the balloon based at least in part on the measured velocity.

In some examples, the method further comprises, based at least in part on the measured velocity of blood flow, determining a desired inflation rate, the desired inflation rate negatively correlated with the measured velocity of blood flow, wherein the received information regarding blood flow across the balloon based at least in part on the desired inflation rate.

In some examples, the rate of inflation of the balloon is based at least in part on the determined desired inflation rate.

In some examples, the method further comprises comparing the measured velocity of blood flow to a plurality of respective predetermined parameters, the desired inflation rate determined based at least in part on an outcome of the comparison to the respective predetermined parameters.

In some examples, the method further comprises, based at least in part on the measured velocity of blood flow, determining a desired inflation time, the desired inflation time period positively correlated with the measured velocity of blood flow, wherein the received information regarding the blood flow across the balloon is based at least in part on the determined desired inflation time.

In some examples, the method further comprises stopping the inflation of the balloon when the determined desired inflation time period ends.

In some examples, the method further comprises comparing the measured velocity of blood flow to a plurality of respective predetermined parameters, the desired inflation time determined based at least in part on an outcome of the comparison to the respective predetermined parameters.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 shows an exemplary delivery apparatus with an inflatable balloon located along a distal end portion.

FIG. 2A shows a side view of an exemplary balloon provided with helical lobes and helical depressions that define helical channels.

FIG. 2B shows a front view in perspective of the balloon of FIG. 2A.

FIG. 3 shows a sectional view along a longitudinal axis of a distal portion of the delivery apparatus.

FIG. 4 shows a cross-sectional view of a central portion of the balloon of FIGS. 2A-2B along a plane orthogonal to the longitudinal axis, defining three helical channels when expanded in a target lumen.

FIG. 5 shows a cross-sectional view of a central portion of one example of a balloon that includes two helical lobes and defines two helical channels.

FIGS. 6A-6B illustrate stages of an exemplary method of the balloon utilized in a valvuloplasty procedure.

FIGS. 7A-7C illustrate stages of an exemplary method of the balloon utilized in a prosthetic valve implantation.

FIG. 8 shows an exemplary balloon expandable prosthetic valve that can be used in combination with balloons of the current disclosure.

FIG. 9 shows an exemplary delivery apparatus with a force sensor attached to a helical lobe of the balloon.

FIG. 10 shows an exemplary configuration of a plurality of force sensors attached to helical lobes of the balloon.

FIG. 11 shows an exemplary configuration of a flow sensor attached to a depressed portion of the balloon.

FIG. 12 shows a high-level flow chart of a method of generating a user notification signal based at least in part on a sensor output.

FIG. 13 shows a high-level flow chart of a method of inflating a balloon with inflation fluid.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be reconfigured or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

It should be understood that the disclosed examples can be adapted to deliver inflatable balloons, and in some implementations, to deliver and implant prosthetic devices expandable by such inflatable balloons, to and/or in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

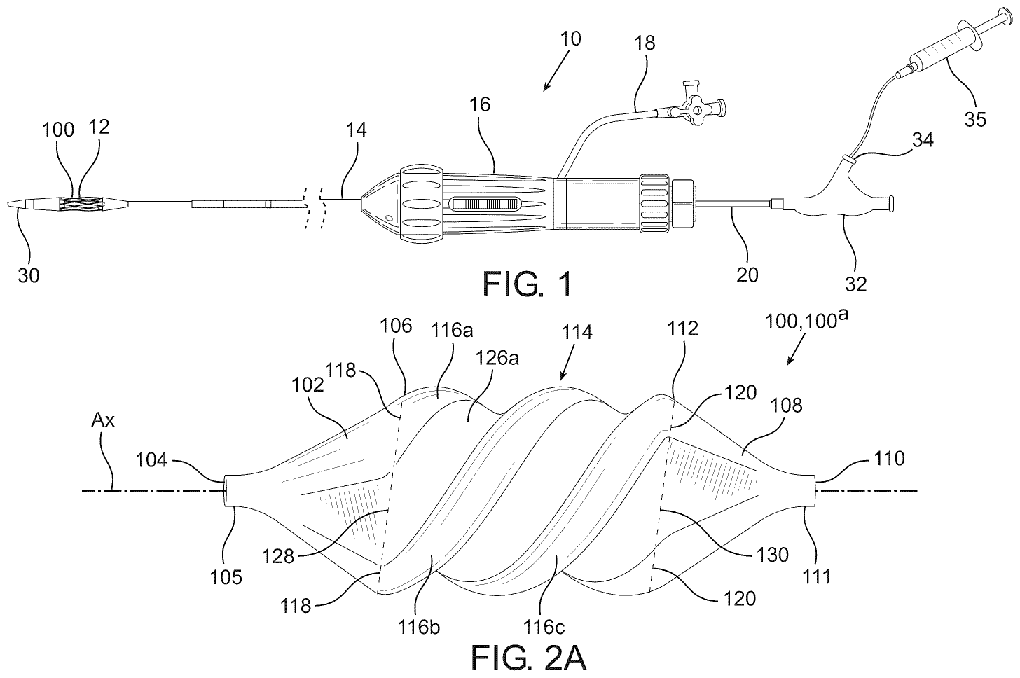

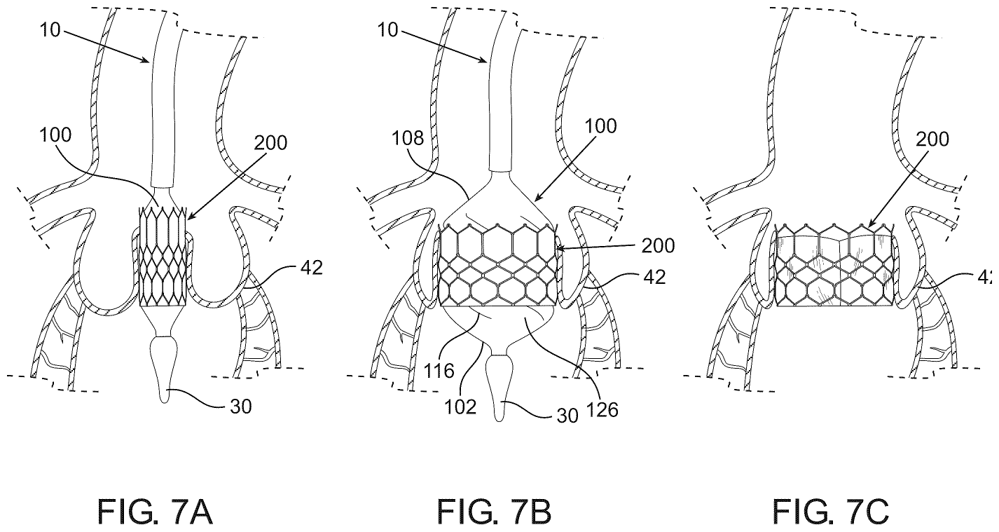

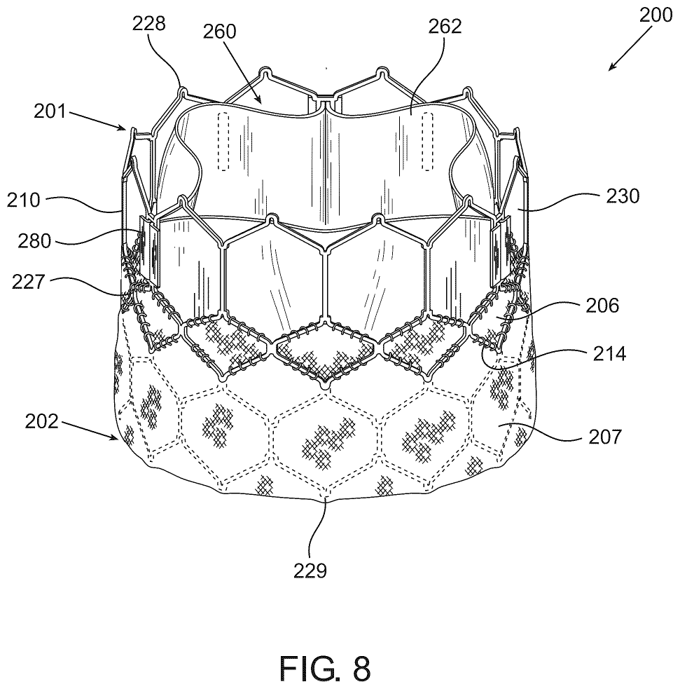

FIG. 1 illustrates a delivery apparatus 10, according to one configuration, adapted to deliver an expandable balloon 100, optionally carrying a balloon expandable prosthetic device 12 thereon. Prosthetic device 12 can be a prosthetic valve, such as the prosthetic valve 200 described below and illustrated with respect to FIGS. 7A-8 or other types of prosthetic valves. It should be understood that the delivery apparatus 10 can be used to implant prosthetic devices other than prosthetic valves, such as stents or grafts, and that delivery apparatus 10 can be used to deliver an expandable balloon that does not necessarily carry an expandable prosthetic device, for example for medical procedures that can include valvuloplasty, pre-ballooning and post-ballooning.

The delivery apparatus 10 can generally include a steerable delivery shaft 14 and a balloon catheter 20 extending through the delivery shaft 14. The delivery shaft 14 and the balloon catheter 20 can be adapted to slide longitudinally relative to each other to facilitate delivery and positioning of a prosthetic device 12 at an implantation site in a patient's body.

The delivery apparatus 10 includes a handle 16 and a balloon catheter 20 having an inflatable balloon 100 mounted on its distal end. The handle 16 can include a side arm 18 having an internal passage which fluidly communicates with a lumen defined by the handle 16. A balloon expandable prosthetic device 12, such as balloon expandable prosthetic valve 200, can be carried in a crimped state over the balloon catheter 20. Optionally, an outer delivery shaft 14 can concentrically extend over the balloon catheter 20. The delivery shaft 14 and the balloon catheter 20 can be adapted to slide longitudinally relative to each other to facilitate delivery and positioning of inflatable balloon 100 and/or a prosthetic device 12 at a treatment site (or implantation site in case of a prosthetic device) in a patient's body.

A nosecone 30 can be mounted at the distal end of the delivery apparatus 10 to facilitate advancement of the delivery apparatus 10 through the patient's vasculature to the site of treatment. In some instances, it may be useful to have nosecone 30 connected to a separate elongated nosecone shaft 26 (shown in FIG. 3) so that nosecone 30 can move independently of other elements of delivery apparatus 10. Nosecone shaft 26 can extend through a lumen 22 of the balloon catheter 20. The balloon catheter 20 and the nosecone shaft 26 can be adapted to slide longitudinally relative to each other.

The balloon catheter 20 can extend through the handle 16 and a proximal portion 32 which can be disposed proximally to the handle 16. The proximal portion 32 can be formed with a fluid passageway 34 that is fluidly connectable to a fluid source for inflating the balloon. The fluid source comprises an inflation fluid 35. The term “inflation fluid”, as used herein, means a fluid (e.g., saline) used for inflating balloon 100. Fluid passageway 34 is in fluid communication with the balloon catheter lumen 22, such as the annular space between the inner surface of balloon catheter 20 and the outer surface of a nosecone shaft 26 disposed therein, such that fluid from the fluid source can flow through fluid passageway 34, through the balloon catheter lumen 22, and into balloon 100 to inflate the same, and optionally deploy prosthetic device 12 if such a device is crimped over the balloon.

In FIG. 1, a prosthetic device 12 is mounted on the balloon 100 and is shown in a crimped state, providing prosthetic device 12 with a reduced diameter for delivery to the heart via the patient's vasculature. As mentioned above, it should be understood that balloon 100 can be configured for delivery to a treatment location without a prosthetic device (such as a prosthetic heart valve) mounted thereon, either for off-balloon delivery of the prosthetic device to a treatment location (as discussed below) or for use of the balloon in a valvuloplasty procedure.

Although the illustrated examples discussed herein refer to the prosthetic device (e.g., prosthetic valve) as being crimped or mounted on the balloon for delivery to the treatment location, it should be understood that the prosthetic device can be crimped or mounted at a location different from the location of balloon (e.g., proximal to the balloon) and repositioned over the balloon at some time before inflating the balloon and deploying the prosthetic device. This off-balloon delivery allows the prosthetic device to be crimped to a lower profile than would be possible if the prosthetic device was crimped on top of the balloon. The lower profile permits the physician to more easily navigate the delivery apparatus (including the crimped prosthetic device) through a patient's vasculature to the treatment location. The lower profile of the crimped prosthetic device can be particularly helpful when navigating through portions of the patient's vasculature which are particularly narrow, such as the iliac artery.

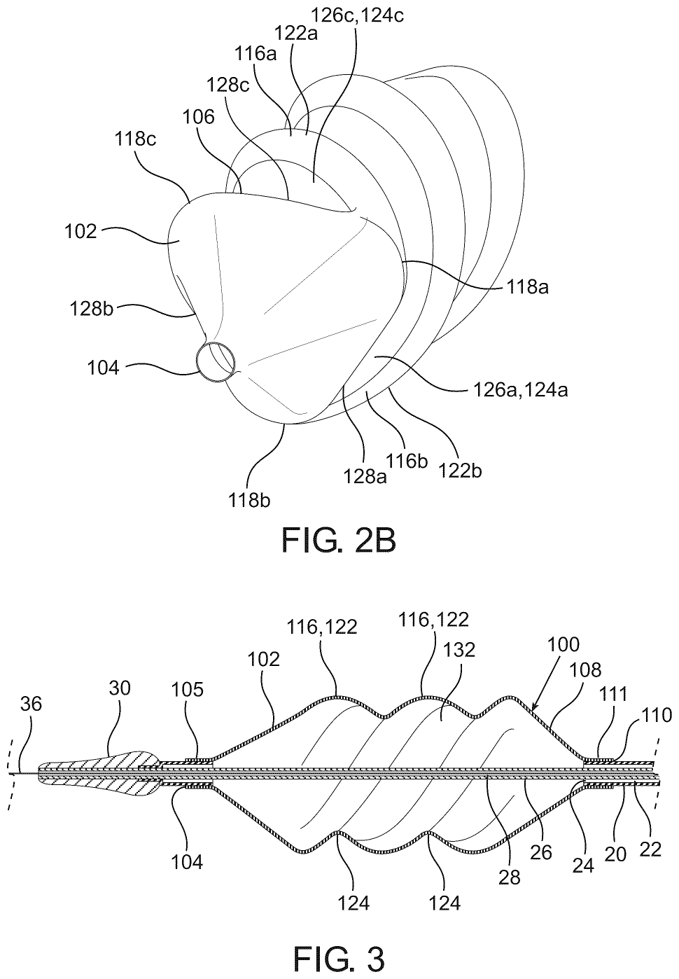

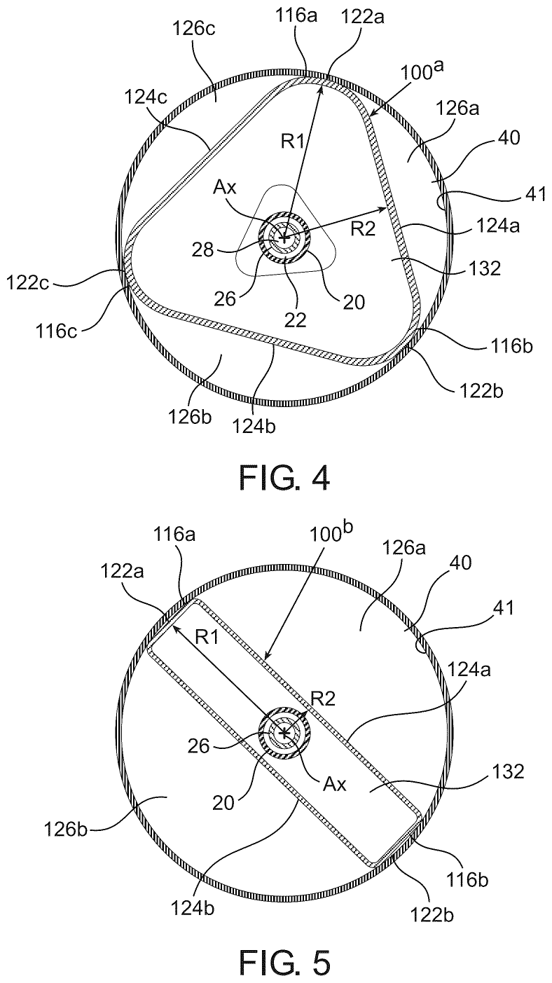

Balloon 100 is configured to transition between a deflated state and an inflated state. FIGS. 2A and 2B show a side view and a front view in perspective of a balloon 100 defining at least two channels 126 in an inflated state thereof. FIG. 3 shows a sectional side-view of a portion of delivery apparatus 10 including the balloon 100 of FIGS. 2A-2B, attached to a balloon catheter 20. FIG. 4 shows a lateral sectional view across the balloon 100 of FIGS. 2A-3. Balloon 100 comprises a leading portion 102, a trailing portion 108, and a central portion 114 extending therebetween. The leading portion 102 and the trailing portion 108 are configured to assume tapering configurations in the inflated state of the balloon. The leading portion 102 extends from a narrower leading portion distal end 104 to a wider leading portion proximal end 106, and the trailing portion 108 extends from a narrower trailing portion proximal end 110 to a wider trailing portion distal end 112, in the inflated state, such that the central portion 114 continuously extends from the leading portion proximal end 106 to the trailing portion distal end 112.

When balloon 100 is inflated, at least two lobes 116 extend along the entire length of the central portion 114, defining a corresponding number (e.g., at least two) of channels 126 extending between adjacent lobes 116 along the entire length of the central portion 114. Each lobe extends between a lobe distal end 118 at the leading portion proximal end 106 and a lobe proximal end 120 at the trailing portion distal end 112. In some examples, the lobes 116 and the channels 126 can be helical in shape. The lobes 116 can, in such examples, spiral along the length of central portion 114 and around the longitudinal axis Ax of the balloon 100, and the channels 126 defined between the adjacent lobes 116 can similarly extend along the length of the central portion 114 and around the longitudinal axis Ax.

FIGS. 2A-4 illustrate an example of a balloon 100ª that includes three lobes 116 defining three channels 126 along the length of the central portion 114 in the inflated state of the balloon. The lobes 116 and the channels 126 are illustrated in FIGS. 2A-4 to be helical in shape. Each of the leading portion 102 and the trailing portion 108 is shown to be pyramid-shaped, defining a substantially triangular cross-sectional shape along its tapering region. The cross-sectional area of each of the leading portion 102 and the trailing portion 108 is greatest at the leading portion proximal end 106 and the trailing portion distal end 112, respectively. The leading portion 102 and the trailing portion 108 can be substantially symmetrical in some examples, such that the cross-sectional area of the leading portion proximal end 106 is equal to the cross-sectional area of the trailing portion distal end 112, in which case it is also the same cross-sectional area at any point along the length of the central portion 114.

In the illustrated example of balloon 100a, the leading portion proximal end 106 has a substantially triangular cross-sectional shape defining three lope distal ends 118a, 118b, 118c at its vertices. Similarly, trailing portion distal end 112 has a substantially triangular cross-sectional shape defining three lobe proximal ends 120a, 120b, 120c at its vertices. Depressed portions 124 are defined between adjacent lobe ends. For example, three distal depressed ends 128 are defined between lobe distal ends 118 at the leading portion proximal end 106, and three proximal depressed ends 130 are similarly defined between lobe proximal ends 120 at the trailing portion distal end 112. Thus, each channel 126 extends from a corresponding distal depressed end 128 to a corresponding proximal depressed end 130. In the example illustrated in FIG. 2B, distal depressed end 128a extends between lobe distal ends 118a and 118b, distal depressed end 128b extends between lobe distal ends 118b and 118c, and distal depressed end 128c extends between lobe distal ends 118c and 118a.

Various exemplary implementations for balloons 100 can be referred to, throughout the specification, with superscripts, for ease of explanation of features that refer to such exemplary implementations. It is to be understood, however, that any reference to structural or functional features of any apparatus, assembly or component, without a superscript, refer to these features being commonly shared by all specific exemplary implementations that can be also indicated by superscripts. In contrast, features emphasized with respect to an exemplary implementation of any apparatus, assembly or component, including balloons 100, referred to with a superscript, may be optionally shared by some but not necessarily all other exemplary implementations. For example, balloon 100ª is an exemplary implementation of balloon 100, and thus includes all of the features described for balloon 100 throughout the current disclosure, except that while balloon 100 can have any number of lobes and channels extending therealong, balloon 100ª is shown to include three helical lobes defining three helical channels therebetween.

As shown in FIG. 3, the balloon 100 can be attached at both ends thereof to proximal and distal portion of the balloon catheter 20. In some examples, the trailing portion 108 can include a proximal attachment section 111 attached to a proximal portion of balloon catheter 20, and the leading portion 102 can include a distal attachment section 105 attached to a distal portion of balloon catheter 20 as shown, or in some examples, to a portion of nosecone 30. The proximal and distal attachment sections 111, 105 can be secured to components of the delivery apparatus 10, such as balloon catheter 20 and/or nosecone 30, by force fitting, heat pressing, welding or a suitable adhesive. The distal attachment section 105 extends from the leading portion distal end 104, such that the leading portion 102 tapers from the end of the distal attachment section 105 to the leading portion proximal end 106. Similarly, the proximal attachment section 111 extends from the trailing portion proximal end 110, such that the trailing portion 108 tapers from the end of the proximal attachment section 111 to the trailing portion distal end 112.

As further shown in FIG. 3, the nosecone shaft 26 extends through the balloon catheter lumen 22, and defines a guidewire lumen 28 extending along the entire length of the nosecone shaft 26 and nosecone 30, through which a guidewire 36 can pass, such that the entire delivery apparatus 10 can be advanced toward the treatment region over the guidewire 36. The diameter of nosecone shaft 26 can be sized such that an annular space is formed within balloon catheter lumen 22 between balloon catheter 20 and nosecone shaft 26 along the length of balloon catheter 20. This annular space can be in fluid communication with one or more openings 24 exposed to an internal cavity 132 of the balloon 100, which can be in fluid communication with a fluid source (e.g., a syringe or a pump) that can inject an inflation fluid 35 (e.g., saline) into cavity 132, optionally via fluid passageway 34. In this way, fluid from the fluid source can flow through the fluid passageway 34, through balloon catheter lumen 22, and into cavity 132 via opening(s) 24 to inflate the balloon 100 and optionally expand and deploy a prosthetic valve 12 when such a device is disposed thereon. For example, the pressure of the fluid within balloon 100 may provide the force that allows the central portion 114 of balloon 100 to dilate the prosthetic device 12 and/or surrounding anatomy. Further, the balloon catheter lumen 22 may be configured to withdraw fluid from the internal cavity 132 through the opening(s) 24 to deflate the balloon 100.

The balloon 100 may be configured to be in a deflated or undeployed state for being positioned in a target lumen of a delivery apparatus 10 and/or of a patient at a target site of treatment, and may be configured to be inflated to a deployed/expanded/inflated state as shown in FIGS. 2A-4. The balloon catheter lumen 22 may be utilized to inflate the balloon 100 to transition the balloon 100 to the inflated or deployed state, and may be utilized to deflate the balloon 100 to transition the balloon 100 to the deflated or undeployed state. Balloon 100 can be formed as unitary components comprising a single cavity 132. The term “unitary”, as used herein with reference to balloon 100, refers to a balloon being formed of a single piece of material (even though several layers of different materials are still allowed), resulting in the balloon defining a single internal cavity.

Nosecone shaft 26, balloon catheter 20 and optional delivery shaft 14 of delivery apparatus 10 can comprise any of various suitable materials, such as nylon, braided stainless steel wires, or a polyether block amide (commercially available as Pebax®). In some examples, balloon catheter 20 and optional delivery shaft 14 of delivery apparatus 10 have longitudinal sections comprising different materials in order to vary the flexibility of the shafts along their lengths. In some examples, nosecone shaft 26 has an inner liner or layer formed of Teflon® to minimize sliding friction with a guide wire 36.

Lobes 116 comprise lobe outer surfaces 122 facing radially outward from longitudinal axis Ax. Lobe outer surfaces 122 are configured to receive and urge against a prosthetic device 12 (i.e., to radially expand the prosthetic device, such as a prosthetic heart valve) and/or configured to urge against an inner surface of a target lumen in which the balloon 100 is disposed, such as a patient lumen (for example, during a valvuloplasty procedure). As shown in FIG. 4, when balloon 100 is inflated (expanded) in a target lumen 40 (which can be a patient lumen, such as an annulus or any other similar orifice or passageway in the body), two or more channels 126 are preferably formed between the corresponding depressed portions 124 and the inner surface of a target lumen 40 of the bounding structure (e.g., prosthetic device 12 or a patient lumen). Thus, each channel 126 is bound between a corresponding depressed portion 124, the neighboring lobes 116 on both sides of the depressed portion 124, and the target lumen 40 of the bounding structure (e.g., prosthetic device 12 or a patient lumen). The lobes 116 can be parallel to each other, resulting in channels 126 which are similarly parallel to each other. The leading and trailing portions 102, 108 of the balloon 100 taper to a narrower diameter from the respective ends of the central portion 114, such that only the lobe outer surfaces 122 at the leading portion proximal end 106 and at the trailing portion distal end 112 can be in contact with the annulus (or other body passage way). Thus, two or more channels 126 can permit blood perfusion through the body passageway between the ends of balloon 100 (i.e., between trailing portion proximal end 110 and leading portion distal end 104) when the balloon is in an inflated/expanded state.

As shown in FIG. 4, the balloon can define, in the inflated state, a major radial distance R1, defined as the maximal distance between the longitudinal axis Ax and the lobe 116, and a minor radial distance R2 defined as the minimal distance between the longitudinal axis Ax and the depressed portion 124, wherein the minor radial distance R2 is less than the major radial distance R1. While the lobes 116 and depressed portions 124 together define a continuous surface of a unitary balloon 100, each lobe 116 can exhibit, at any cross-section of the balloon at a plane perpendicular to axis Ax (such as the plane of view in FIG. 4), a lobe curvature which is greater that a depressed portion curvature exhibited by any of the depressed portions 124. In the illustrated example, the lobes 116 are shown to exhibit curved convex cross-sectional shape, while the depressed portions 124 are shown to be linear or slightly concave. In alternative designs, both the lobes 116 and the depressed portions 124 can exhibit similar curvatures, such as both being substantially linear, forming step-like configuration there-between (not shown) with the lobes 116 offset radially away (to a distance R1) relative to the depressed portions 124.

The balloon 100 can have the same cross-sectional shape along the entire length of central portion 114, wherein the cross-sectional shape (i.e., along a plane perpendicular to axis Ax) is angularly offset around axis Ax with respect to the shape of a preceding or succeeding cross section along the axis Ax. In some examples, the curvatures of some of the lobes 116 are identical to each other, and the curvatures of some of the depressed portions 124, are identical to each other in any cross-section across a plane perpendicular to the axis Ax, along the length of the balloon's central portion 114. In some examples, the curvatures of all of the lobes 116 are identical to each other, and the curvatures of all of the depressed portions 124, are identical to each in any cross-section across a plane perpendicular to the axis Ax, along the length of the balloon's central portion 114. In some examples, the same major radial distance R1 is identical for some of the lobes 116, and the same minor radial distance R2 is identical for some of the depressed portions 124. In some examples, the same major radial distance R1 is identical for all of the lobes 116, and the same minor radial distance R2 is identical for all of the depressed portions 124.

Both the lobes and the depressed portions can be shaped to curve along any cross-section in a substantially smooth manner (i.e., continuous to each other), without forming any acute or right angles therebetween along the outer surface of the balloon. This can advantageously reduce the likelihood of flow disturbances along the channels 126, to mitigate the risk of thrombosis that may otherwise occur when flow disturbances, such as stagnation zones and/or local vortices, are introduced.

FIG. 5 shows an exemplary balloon 100b that includes two lobes 116 with two depressed portions 124 extending therebetween, defining two channels 126 between the depressed portions 124 and the surrounding walls of target lumen 40. The lobes 116 and channels 126 of balloon 100b can be helical in shape. The balloon 100b is an example of balloon 100, and thus includes all of the features described for balloon 100 throughout the current disclosure, except that while balloon 100 can include any number of lobes and channels, balloon 100b is shown to include two lobes defining two channels therebetween. For example, balloon 100b can be similar to the balloon 100ª, except that while balloon 100ª includes three helical lobes with three depressed portions extending therebetween, defining three helical channels, balloon 100b includes two helical lobes 116 with two depressed portions 124 extending therebetween, defining two helical channels 126. While the cross-sectional shape of the inflated balloon 100b at any point along axis Ax of the central portion 114, as well as cross-sectional shapes along tapering portions of the leading and trailing portions 102, 108 can be substantially rectangular in shape as in the illustrated example, other shapes that include two lobes, such as an ellipse, a peanut-shaped sectional configuration, and the like, are contemplated. As shown, the major radial distance R1 extending from axis Ax to each lobe 116 is greater than the minor major radial distance R2 extending from axis Ax to each depressed portion 124. While balloons 100b with two lobes defining two channels are illustrated in FIG. 5, and balloons 100ª with three lobes defining three channels are illustrated in FIGS. 2A-4, it is to be understood that some examples can include more than three lobes and/or channels.

Conventional inflation procedures, particularly for replacement of a native aortic valve, are usually performed under rapid pacing. Rapid pacing is a technique that involves electrical stimulation of the heart using pacemaker leads inserted into the heart. The heart rhythm of the patient is then accelerated to over 180 bpm which in fact causes the heart to flutter and thus not to effectually contract. While rapid pacing may help in toleration of the native valve's occlusion, the added procedure involves added risk, and a small number of patients do not tolerate accelerated pacing very well. In some rare instances, there can be long term myocardial damage due to extended rapid pacing. Advantageously, by providing balloons 100 with channels 126 that allow continuous blood perfusion therealong, a physician can have additional time to perform the interventional procedures, such as valvuloplasty or prosthetic device implantation that allow the physician to deploy (or collapse) the prosthetic device, while the risk of significant adverse effects due to blood occlusion through the orifice or passageway can be reduced. Such procedures can be performed, in some examples, without rapid pacing.

Various types of previously disclosed expansion devices, described for example in U.S. Pat. No. 9,707,078, include a plurality of similarly shaped or differently shaped separate balloons, such as a plurality of outer balloon members disposed around an inner balloon member or an outer balloon member helically wrapped around an inner balloon member, configured to form voids in an inflated state thereof allowing blood perfusion therethrough. Additional previously disclosed configuration can include an elongated or tubular balloon helically wound around the longitudinal axis of the delivery apparatus. In contrast, the balloon 100 described herein is an unwrapped/unwound balloon, meaning that the channels are not formed by wrapping a lender elongated balloon around a shaft or catheter, such as around a balloon catheter or a nosecone shaft. Such unwrapped balloons 100 are advantageous over wrapped balloon configurations, since a wrapped balloon will include two wall layers disposed on both sides of a central shaft or catheter, while an unwrapped/unwound balloon can be deflated to a lower profile resulting in a single wall balloon layer disposed on each side of the longitudinal axis. This lower profile permits the surgeon to more easily navigate the delivery apparatus (including deflated balloon 100) through a patient's vasculature to the treatment location. The lower profile of the deflated balloon is particularly helpful when navigating through portions of the patient's vasculature which are particularly narrow, such as the iliac artery.

Additional alternative prior art configurations may include balloons configured to form helical channels around peripheries thereof by additional biasing members. For example, some balloon configurations can include outer constriction members, such as helical wires or mold bands disposed around the outer surface of the balloons, configured to restrict balloon expansion to the portions disposed between such members. Other balloon configurations can include internal biasing means, such as a spring coil disposed within the balloon's cavity, configured to transition from an elongated compacted configuration during delivery, to an outwardly biased configuration pushing against the balloon wall to force it into the helical configuration. The balloons 100 of the current disclosure are devoid of any internal biasing means or external constriction members, since such additional elements can add to the crimped profile of the apparatus during delivery. Other prior art configurations may include portions of the balloon having an outer surface thereof configured to contact an outer surface of an internal shaft, such as a nosecone shaft, which serves to form a flow channel around the balloon in its inflated state. As before, for the sake of reducing the crimped profile, no part of the outer surface of the central portions 114 of balloon 100 as disclosed herein is exposed to an outer surface of the nosecone shaft.

Additional previously disclosed expansion devices can include a balloon having a plurality of projections extending radially outward from a main body thereof, with the projections defining grooves in-between configured to permit blood flow between both ends of the balloon. Such designs usually include projections extending from opposite sides of the balloon across the perimeter of the balloon, eventually occupying a cross-sectional diameter which is equal to the distance between the radial end surfaces of opposing projections. In contrast, the perimeter of the currently disclosed balloon 100 at any cross-section along its central portion 114 does not span the perimeter of the bounding circle, resulting in a cross-sectional area (as shown in the examples illustrated in FIGS. 4 and 5, for example) that can be significantly less than that of balloons shaped to include radially extending projections, which also serves to allow a significantly lower crimped profile of the balloon 100.

Balloon 100 may be made of one polymer, or use several layers or a mix of different polymers. Polymers such as Nylon, PEBAX, PET, parylene and/or polyurethane may be used to make the balloon 100. Various techniques can be utilized for manufacturing balloon 100. For example, the balloon 100 may be fabricated by blow molding, wherein the mold can be shaped to provide the desired outer shape of the balloon 100, including the plurality of lobes and depressed portions.

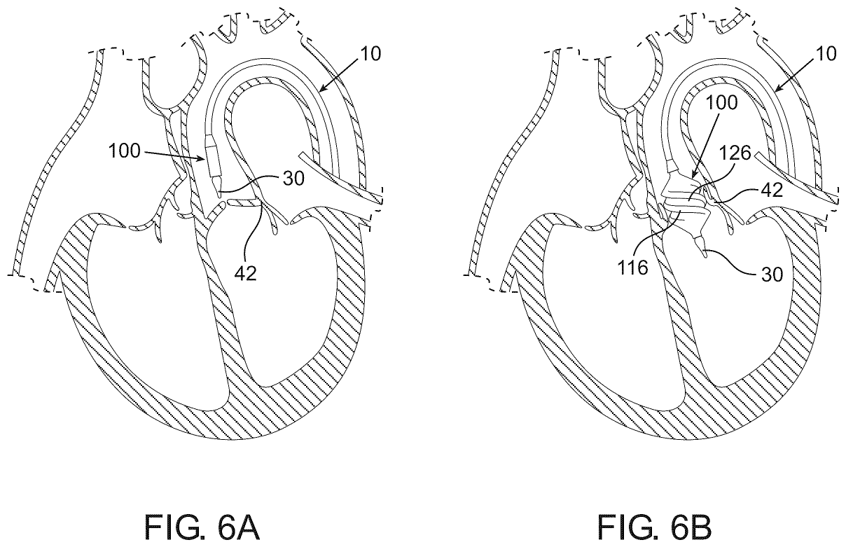

FIGS. 6A-6B illustrate an exemplary method of utilizing balloons 100 to expand an orifice or passageway of the body, such as during valvuloplasty procedures. That is, the expansion of the balloon 100 can be done in the illustrated method without a prosthetic device crimped thereon in a valvuloplasty procedure. Methods disclosed herein may vary from the steps shown in FIGS. 6A-6B. FIG. 6A illustrates a step in a method of dilating a native orifice or passageway in a patient's body, such as the aortic heart valve. A delivery apparatus, such as delivery apparatus 10 illustrated in FIG. 1, may be utilized to approach the native heart valve or other orifice in the patient's body. The delivery shaft 14 may be deflected to allow the balloon 100 to approach the native aortic heart valve 42 through the aortic arch. Referring to FIG. 6B, the balloon 100 can be inflated to press against the native annulus and/or the native heart valve leaflets to expand the aortic annulus. While described and illustrated with respect to a native aortic heart valve, it is to be understood that balloons 100 can be similarly utilized to press against and expand other native heart valves, such as a mitral, tricuspid, or pulmonary heart valve, or any other orifice, such as stenotic portions along an artery of the patient.

FIGS. 7A-7C illustrate a method of deploying a prosthetic heart valve 200 within a native aortic annulus. Referring to FIG. 7A, a delivery apparatus 10 is shown delivering a prosthetic heart valve 200 in a collapsed configuration. Delivery apparatus 10 can deliver prosthetic valve 200 to the treatment location using known procedures. The prosthetic valve can be delivered either through a transfemoral or transapical approach.

Prosthetic valve 200 can be mounted on balloon 100. Prosthetic valve 200 is maneuvered within a native aortic valve annulus 42 for deployment using delivery apparatus 10. Referring to FIG. 7B, balloon 100 is inflated by injecting inflation fluid 35 into the cavity 132 of balloon 100. Blood is allowed to flow during systole through channels 126 from the left ventricle toward the aorta. After prosthetic valve 200 is deployed within the native aortic annulus 42, balloon 100 can be deflated and removed from the aortic annulus (FIG. 7C). While a prosthetic valve 200 is described and illustrated in FIG. 7A-7C to expand within a native aortic heart valve, it is to be understood that prosthetic valve 200 can be similarly deployed in other native heart valves, such as a mitral, tricuspid, or pulmonary heart valve. Moreover, while balloon 100 is shown in combination with a prosthetic valve 200 in FIGS. 7A-7C, it is to be understood that the method can be similarly utilized for deployment of other prosthetic devices by inflatable balloons 100, such as stents that can be deployed in stenotic arteries. It is to be understood that balloons 100 can be similarly utilized to press against and expand other native heart valves, such as a mitral, tricuspid, or pulmonary heart valve.

The term “prosthetic valve”, as used herein, refers to any type of a balloon expandable prosthetic valve deliverable to a patient's target site over a catheter, which is radially expandable and compressible between a radially compressed, or crimped, configuration, and a radially expanded configuration. Thus, the prosthetic valve can be crimped on or retained by an implant delivery apparatus (such as delivery apparatus 10 shown in FIG. 1) in the radially compressed configuration during delivery, and then expanded to the radially expanded configuration once the prosthetic valve reaches the implantation site. A prosthetic valve of the current disclosure may include any prosthetic valve configured to be mounted within the native aortic valve, the native mitral valve, the native pulmonary valve, and the native tricuspid valve.

FIG. 8 shows a view in perspective on an exemplary prosthetic valve 200 that can be expanded by balloon 100, illustrated in an expanded configuration. The prosthetic valve 200 can comprise an outflow end 201 and an inflow end 202. In some instances, the outflow end 201 is the proximal end of the prosthetic valve 200, and the inflow end 202 is the distal end of the prosthetic valve 200. Alternatively, depending for example on the delivery approach of the valve, the outflow end can be the distal end of the prosthetic valve, and the inflow end can be the distal end of the prosthetic valve. The term “outflow”, as used herein, refers to a region of the prosthetic valve through which the blood flows through and out of the prosthetic valve 200. The term “inflow”, as used herein, refers to a region of the prosthetic valve through which the blood flows into the prosthetic valve 200.

The prosthetic valve 200 comprises an annular frame 210 movable between a radially compressed configuration and a radially expanded configuration, and a valvular structure 260 mounted within the frame 210. The frame 210 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 210 can be crimped to a radially compressed configuration on the balloon catheter 20, and then expanded inside a patient by the inflatable balloon 100.

In the example illustrated in FIG. 8, the frame 210 is an annular, stent-like structure comprising a plurality of intersecting struts 214. A strut 214 may be any elongated member or portion of the frame 210. The frame 210 can include a plurality of strut rungs that can collectively define one or more rows of cells 230. The frame 210 can have a cylindrical or substantially cylindrical shape having a constant diameter from the inflow end 202 to the outflow end 201 as shown, or the frame can vary in diameter along the height of the frame, as disclosed in U.S. Pat. No. 9,155,619, which is incorporated herein by reference.

The end portions of the struts 214 are forming apices 228 at the outflow end 201 and apices 229 at the inflow end 202. The struts 214 can intersect at additional junctions 227 formed between the outflow apices 228 and the inflow apices 229. The junctions 227 can be equally or unequally spaced apart from each other, and/or from the apices 228, 229, between the outflow end 201 and the inflow end 202.

At least some of the struts can be pivotable or bendable relative to each other, so as to permit frame expansion or compression. For example, the frame 210 can comprise a single piece of material, such as a metal tube, via various processes such as, but not limited to, laser cutting, electroforming, and/or physical vapor deposition, while retaining the ability to collapse/expand radially in the absence of hinges and like.

A valvular structure 260 can include a plurality of leaflets 262 (e.g., three leaflets), positioned at least partially within the frame 210, and configured to regulate flow of blood through the prosthetic valve 200 from the inflow end 202 to the outflow end 201. While three leaflets 262 configured to collapse in a tricuspid arrangement are shown in the illustrated example, it will be clear that a prosthetic valve 200 can include any other number of leaflets 262. The leaflets 262 can be made from, in whole or part, biological material (e.g., pericardium), bio-compatible synthetic materials, or other such materials. Further details regarding transcatheter prosthetic heart valves, including the manner in which the valvular structures 260 can be coupled to the frame 210 of the prosthetic valve 200, can be found, for example, in U.S. Pat. Nos. 6,730,118, 7,393,360, 7,510,575, 7,993,394, 8,652,202, and 11,135,056, all of which are incorporated herein by reference in their entireties.

The leaflets 262 define a non-planar coaptation plane (not annotated) when free outflow edges thereof co-apt with each other to seal blood flow through the prosthetic valve 200. Adjacent leaflets 262 can be secured to one another to form commissures 280 of the valvular structure 260, which can be secured, directly or indirectly, to structural elements connected to the frame 210 or integrally formed as portions thereof, such as commissure posts, commissure windows, and the like. When the leaflets 262 are coupled to the frame and to each other, the lower edge of the resulting valvular structure 260 desirably has an undulating, curved scalloped shape. By forming the leaflets with this scalloped geometry, stresses on the leaflets 262 are reduced which, in turn, improves durability of the prosthetic valve. Moreover, by virtue of the scalloped shape, folds and ripples at the belly of each leaflet, which can cause early calcification in those areas, can be eliminated or at least minimized. The scalloped geometry also reduces the amount of tissue material used to form the valvular structure, thereby allowing a smaller, more even crimped profile at the inflow end of the valve.

In some examples, the prosthetic valve can further comprise at least one skirt or sealing member. FIG. 8 shows an example of a prosthetic valve 200 that includes an inner skirt 206, which can be secured to the inner surface of the frame 210. Such an inner skirt 206 can be configured to function, for example, as a sealing member to prevent or decrease perivalvular leakage. An inner skirt 206 can further function as an anchoring region for valvular structure 260 to the frame 210, and/or function to protect the leaflets 262 against damage which may be caused by contact with the frame 210, for example during valve crimping or during working cycles of the prosthetic valve 200. Additionally, or alternatively, the prosthetic valve 200 can comprise an outer skirt 207 mounted on the outer surface of frame 210, configure to function, for example, as a sealing member retained between the frame 210 and the surrounding tissue of the native annulus against which the prosthetic valve is mounted, thereby reducing risk of paravalvular leakage past the prosthetic valve 200.

Any of the inner skirt 206 and/or outer skirt 207 can be made of various suitable biocompatible materials, such as, but not limited to, various synthetic materials (e.g., PET) or natural tissue (e.g., pericardial tissue). In some cases, the inner skirt 206 can be formed of a single sheet of material that extends continuously around the inner surface of frame 210. In some cases, the outer skirt 207 can be formed of a single sheet of material that extends continuously around the outer surface of frame 210.

In some examples, a delivery apparatus 10 comprising an inflatable balloon 100 and a prosthetic valve assembled thereon, can be packaged in a sterile package that can be supplied to end users for storage and eventual use. In some examples, the leaflets of the prosthetic valve (can be made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 200 and the delivery apparatus 10 can be free of any liquid. Methods for treating tissue leaflets for dry storage are disclosed in U.S. Pat. Nos. 8,007,992 and 8,357,387, both of which documents are incorporated herein by reference.

When inflating a balloon in valvuloplasty procedure, it is desirable to avoid applying excessive force to calcifications present in the artery. Additionally, when implanting a prosthetic device, such as balloon expandable valve 200, it is desirable to expand the valve to a maximum size allowed by the patient's anatomical considerations, in order to avoid paravalvular leakage or other unfavorable hemodynamic phenomena across the valve that may be associated with a mismatch between the valve's expansion diameter and the surrounding tissue, while mitigating the risk of annular rupture that may result from over-expansion.

The balloon 100 can be provided, in some examples, with at least one sensor 150, such as a force or pressure sensor 152, configured to provide feedback regarding the force applied by the balloon 100 on the surrounding anatomy and/or on the prosthetic device 12 expanded by the balloon.

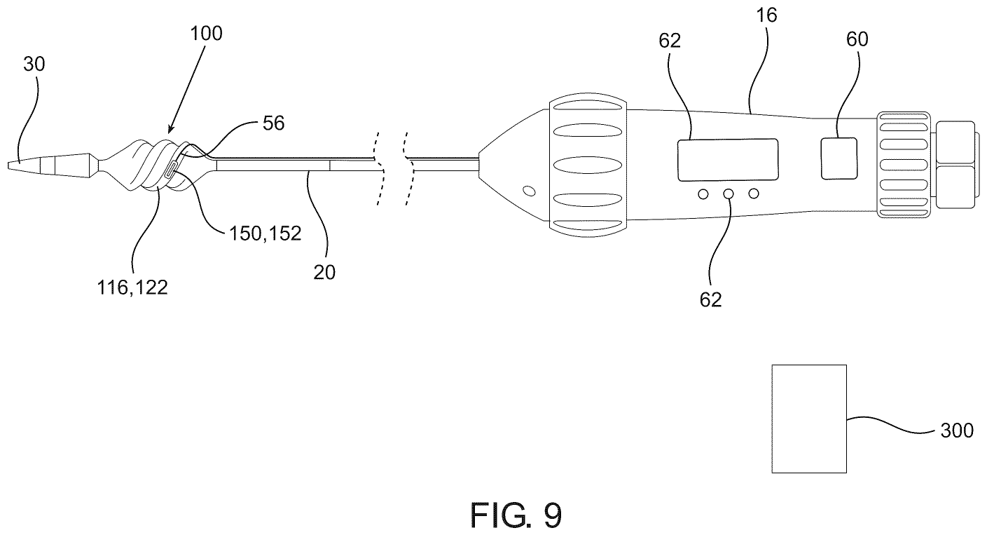

FIG. 9 shows an example of a delivery apparatus 10 with at least one sensor 150 attached to the central portion 114 of the balloon 100. The delivery apparatus 10 can further include one or more optional communication devices 56, a sensor data unit 60, and one or more user output devices 62. The term “communication device”, as used herein, means any device that allows communication therethrough, passively and/or actively. In some examples, this includes wires, optical fibers or wireless communication terminals. In some examples, the one or more user output devices 62 comprise respective visual and/or auditory informative elements configured to generate a visual and/or auditory information, such as, such as a display, LED lights, speakers (not shown) and the like. These options are not limiting, and other feedback can also be provided to a user or operator of delivery apparatus 10 by the one or more user output devices 62.

In some examples, the sensor data unit 60 is in communication with an external system 300. In some examples, external system 300 comprises a processor and a memory (not shown). The memory has stored therein a plurality of instructions, which when run by the processor causes the processor to perform a plurality of predetermined functions, as will be described below. In some examples, communication between sensor data unit 60 and external system 300 is performed via dedicated antennas and/or connection to various networks.

In some examples, the sensor data unit 60 comprises one or more processors and a memory, the memory having a plurality of instructions stored therein. When the one or more processors reads the plurality of instructions, the plurality of instructions cause the one or more processors to perform the functions of the sensor data unit 60. In some examples, the sensor data unit 60 is implemented on a microcontroller, with one or more peripherals of the microcontroller in communication with the at least one sensor 150.

In some examples, the at least one sensor 60 comprises at least one force sensor 152. The term “force sensor”, as used herein, means any sensor that senses the magnitude of a force, or pressure, applied thereto. It is particularly noted that anywhere in the disclosure (in the description and/or claims) where the term “force sensor” is used, this can include a pressure sensor. The force sensor 152 can comprise, in some examples: a piezoresistive sensor, such as a strain gauge or a strain gauge bridge, the resistance of the piezoresistive sensor being a respective predetermined function of the force applied thereto; a piezoelectric sensor, the voltage at the output of the piezoelectric sensor being a respective predetermined function of the force applied thereto; a capacitive sensor, the capacitance of the capacitive sensor being a respective predetermined function of the force applied thereto; and/or an optical sensor, the optical interferometry of the optical sensor being a respective predetermined function of the force applied thereto. In some examples, the at least one sensor 150 comprises a wide diameter pressure sensor.

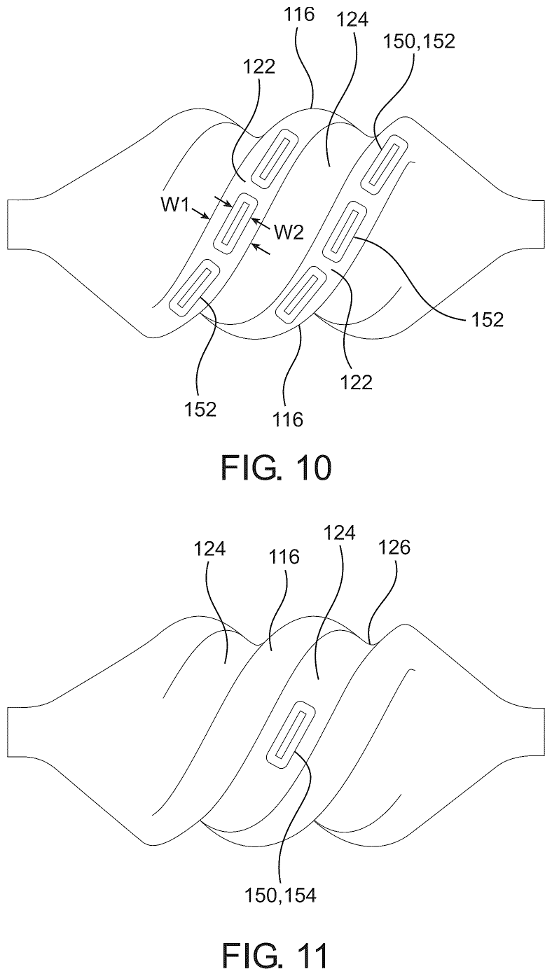

FIG. 9 illustrates an exemplary configuration with a single force sensor 152 attached to the balloon 100. A force sensor 152 will be preferably situated on a portion of at least one of the lobes 116, and more specifically, attached to the lobe outer surface 122, configured to contact the inner surface of target lumen 40 against which the balloon is inflated, such as anatomical walls of a patient lumen (e.g., walls of an annulus or a blood vessel) or a prosthetic device 12 (e.g., prosthetic valve 200) disposed around the balloon 100. FIG. 10 shows an optional configuration in which a plurality of force sensors 152 are attached to the lobe outer surface(s) 122. For simplicity, communication devices 56 are not illustrated in FIG. 10. In one example, a plurality of force sensors 152 can be attached to different portions of the lobe outer surface 122 of the same lobe 116. In some examples, at least one sensor 150 is attached to more than one lobe 116.

In some examples, a balloon 100 designed to allow attachment of one or more sensor(s) 150, such as force sensor(s) 152, to its lobes(s) 116, is provided with a width W1 of the lobe outer surface(s) 122 which is equal to or greater than the width W2 of sensor(s) 152, so as to allow sufficient surface area along which sensor(s) 152 can be situated. The width W1 is defined as the width of a lobe outer surface 122 configured to contact the inner surface 41 of a target lumen 40 such as a patient lumen (for example, annulus 42) or a prosthetic valve 200 disposed on balloon 100 when inflated. In some examples, the lobe outer surface 122 of any lobe 116 of balloon 100 is shaped to remain relatively flat or uncurved at all times (i.e., both during the deflated and inflated states of the balloon), along at least a width W1 of the lobe outer surface 122. This can serve to ensure proper attachment of the sensor(s) 152 to the lobe(s) 116, and/or serve to ensure proper contact with an exterior surface against which the balloon is inflated, such as the inner surface 41 of a target lumen 40 (e.g., patient lumen such as annulus 42) or a prosthetic valve 200 disposed on balloon 100.

In some examples, the at least one communication device 56 is configured to allow: electrical communication via a conductive material, such as a wire; and/or optical communication, e.g., via an optical fiber. In some examples, the at least one sensor 150 is in communication with sensor data unit 60. Only one communication device 56 is illustrated in FIG. 9, however this is not meant to be limiting in any way. In some examples, the at least one sensor 150 is in wireless communication with sensor data unit 60. In one example, a plurality of sensors 150 are separated into a plurality of sets, each set of sensors 150 in communication with sensor data unit 60 via a respective communication device 56. In such an example, as will be described below, the operation of sensors 150 is multiplexed such that only one respective sensor 150 of each set outputs data each time.

In one example, the at least one sensor 150 is operated by sensor data unit 60 such that the sensing of sensor 150 is performed in cooperation with sensor data unit 60. For example, where the at least one force sensor 152 comprises a strain gauge bridge, sensor data unit 60 applies a predetermined excitation voltage at the input leads of the bridge and measures the voltage at the output leads of the bridge. Sensor data unit 60 then determines the applied force, or pressure, from the measured output voltage. In some examples, the at least one sensor 150 comprises dedicated circuitry for operation and sensor data unit 60 receives the measured data from the sensor 150. In some examples, the at least one sensor 150 is in wireless communication with an external computing device.

As inflatable balloon 100 presses against the walls of target lumen 40 (such as against anatomical wall of a patient lumen or against a prosthetic device 12 which in turn is pressed against a patient lumen), a force is applied to sensor(s) 150. In an example where sensor(s) 150 comprises at least one force sensor 152, the at least one force sensor 152 measures the applied force and/or pressure. Based at least in part on the received measurements of sensor(s) 152, sensor data unit 60 determines the pressure between inflatable balloon 100 and the walls of target lumen 40.

In some examples, sensor data unit 60 determines an average, or other predetermined function, of the measurements of sensors 150 in order to achieve a more accurate measurement of the pressure between balloon 100 and the walls of target lumen 40. In some examples where at least one sensor 150 comprises a wide diameter pressure sensor, an accurate measurement of the pressure between balloon 100 and the walls of target lumen 40 is provided.

As described above, in one example the measurements of sensor(s) 150 are performed in cooperation with sensor data unit 60. Alternatively, the measurements of sensor(s) 150 are performed by dedicated circuitry of sensor(s) 150 and transmitted to sensor data unit 60.

In one example, where at least one sensor 150 comprises a plurality of force sensors 152, such as a grid of force sensors 152, sensor data unit 60 determines a map of the forces applied to balloon 100. The term “map of forces”, as used herein, means a representation, graphical or otherwise, of the locations and magnitudes of forces and/or pressures applied to balloon 100. In one example, sensor data unit 60 outputs an indication of the determined map. In one further example, outputting the indication of the determined map comprises controlling the display of user output device 62, or an external user display, to display the determined map.

In one example, sensor data unit 60 identifies any areas in the map of forces which exhibit increased forces applied thereto. These areas may be indicative of calcifications being pressed against balloon 100. In one further example, the increased forces are identified by comparing the forces to one or more predetermined threshold values. Alternatively, or additionally, the increased forces are identified in relation to an average, or other function, of the identified force values from all or a portion of sensors 152. In one example, the magnitude of force applied by a calcification is determined by subtracting from the measured force, applied by the calcification, an average of the forces measured by the rest of sensors 152.

In one example, based at least in part on an indication of an area exhibiting increased force or pressure, sensor data unit 60 outputs a signal indicative of the presence, location and/or applied magnitude of force or pressure of the one or more calcifications. In some examples, based at least in part on the determined magnitude of force applied by the one or more calcifications, sensor data unit 60 determines a maximum inflation pressure value allowed for inflation of balloon 100 and outputs a respective signal indicating the determined maximum inflation pressure value. In one further example, sensor data unit 60 monitors the determined magnitude of pressure applied by the calcifications and the pressure applied by the uncalcified portions of patient lumen 40. The maximum inflation pressure value can then be determined by identifying the pressure applied by the uncalcified portions of patient lumen 40 when the pressure applied by one or more calcifications reaches a predetermined value.

In some examples, based at least in part on the determined magnitude of force applied by the one or more calcification, sensor data unit 60 determines an appropriate orientation for the prosthetic heart valve. For example, in one example, sensor data unit 60 determines an appropriate orientation such that predetermined portions of the prosthetic device 12 won't come in contact with the identified calcification. In one example, sensor data unit 60 identifies the location of the calcification in relation to predefined locations on balloon 100, these predefined locations corresponding to the predetermined portions of the prosthetic device 12. Sensor data unit 60 thus determines the appropriate orientation for the prosthetic device 12 in relation to the orientation of balloon 100. In one example, sensor data unit 60 outputs an indication of the determined orientation.

In some examples, based at least in part on the determined magnitude of force applied by the one or more calcifications, sensor data unit 60 determines whether implantation of a prosthetic device 12 at the anatomical location where balloon 100 is currently located is viable. Particularly, if it is not viable, a different type of prosthetic device can be implanted, such as a mechanically expandable prosthetic valve. In one example, sensor data unit 60 outputs an indication of the determined viability. In one example, viability is determined by determining the maximum pressure values applied by one or more calcifications. If the determined maximum pressure exceeds a predetermined pressure threshold, implantation of the balloon expandable device is considered unviable.

Sensor(s) 152 can be utilized to provide real-time pressure measurement during ongoing inflation of balloon 100. In some cases, increase in balloon and/or prosthetic device diameter is also simultaneously measured, such as by additional appropriate sensor(s) or an estimate from real-time imaging such as Fluoroscopy. In one example, sensor data unit 60 compares a predetermined function of the difference between the increase in pressure and the increase in diameter to a predetermined threshold. In one example, the predetermined function is a derivative of a curve plotted from: the increase values of the pressure and diameter; and/or the absolute values thereof.

It is known from material science that stress-strain curves describe the relationship between stress and strain, and can be obtained by gradually applying a load (i.e., force) to a material and measuring the deformation caused thereto as a result of the applied load. Certain materials exhibit a behavior, in which the strain initially increases in a proportional ratio to the increase in the stress applied to the material (the linear elastic region). After a certain critical point (e.g., yield strength), the stress increase can cause the material to undergo plastic deformation and/or to suffer failure (e.g., fracture).

It is contemplated that arterial and annular tissues (e.g., at a native heart valve) can exhibit certain similar behaviors, as described by stress-strain curves. For example, upon the initial application of a radial expanding force (i.e., stress) to the tissue, and more specifically to the annulus, the annular diameter can increase in a proportional ratio to the increase in the radial force applied thereto (an elastic region). After reaching a certain critical diameter, the tissue is expanded or stretched beyond its physiological limit, and therefore increasing the application of radial forces thereto can cause the tissue to sustain irreversible plastic deformation and/or suffer critical damage (e.g., rupture).

As described above, in some examples each of a plurality of sets of sensors 150 is in communication with sensor data unit 60 via a respective communication device 56. In such examples, communication between sensors 150 and sensor data unit 60 is multiplexed. For example, at each of a plurality of time points a respective sensor 150 of each set transmits data to sensor data unit 60. In further examples, the measurements are performed by the respective sensors 150 at the respective time points, i.e. at each time point only a respective sensor 150 of each set performs a measurements. In additional examples, the measurements are performed continuously, and the communication with sensor data unit 60 is performed one sensor at a time.

The mapping and/or imaging of the balloon/annulus described herein may be performed in real-time or near real-time during a balloon insertion and inflation procedure, which may be executed for a valvuloplasty or other repair procedure, or which may be executed prior to a prosthetic valve deployment (e.g., prior to a transcatheter aortic valve implantation procedure) in some examples. The above-described mapping may be performed in order to model the anatomy to provide measurements for use during a later deployment of the prosthetic valve (e.g., a distance to the annulus, an amount of inflation to use, a size of the prosthetic valve to use to fit the annulus, etc.) and/or to prepare the patient for the later deployment of the prosthetic valve (e.g., to identify obstacles in the annulus or the pathway to the annulus that may be targeted for clearing prior to deployment of the prosthetic valve). In some examples, the mapping and/or imaging of the balloon/annulus may be performed during deployment of a prosthetic valve in order to provide real-time feedback of the deployment status and allow optimization of the deployment size with the specific anatomy.

In some examples, as illustrated in FIG. 11, the at least one sensor 150 comprises at least one flow sensor 154. At least one flow sensor 154 can be secured to at least one depressed portion 124 of the balloon 100, to measure blood flow through the corresponding channel 126. In some examples, at least one flow sensor 154 is attached to each of a plurality of depressed portions 124, to measure flow through each of the channel 126 in the inflated state of balloon 100. Such data can provide an indication whether any or all channels 126 allow adequate blood perfusion therealong.

Since the outer surface of balloon 100 exhibits peaks and depressions in an inflated state thereof, the type of the sensors 150 dictates the appropriate attachment region over the surface of balloon 100. For example, pressure or force sensors 152 can be attached to lobe outer surfaces 122 of lobes 116 to ensure proper contact with a target lumen 40, such as a patient lumen and/or prosthetic device 12, when balloon 100 is inflated, while positioning such sensors along other regions of the balloon, such as along depressed portions 124, will not yield adequate measurements since such regions are not pressed against any outer surface surrounding the balloon. In contrast, in some examples, flow sensors 154 should be attached only to surfaces of the channels 126, such as depressed portions 124, to ensure proper exposure to blood flow through channels 126 when balloon 100 is inflated, while positioning such sensors over lobe outer surfaces 122 will not yield adequate measurements since such surfaces are not exposed to the blood flow around the balloon. In some examples, the at least one sensor 150 includes both one or more force sensor(s) 152 attached to lobe outer surface(s) 122 of lobe(s) 116, and at least one flow sensor(s) 154 attached to depressed portion(s) 124 of channel(s) 126.

In some examples, the at least one flow sensor(s) 154 comprises at least one ultrasonic flow sensor. The term “ultrasonic flow sensor”, as used herein, means a flow sensor based on ultrasound detection. For example, as known to those skilled in the art, an ultrasonic transducer generates an ultrasonic wave directed at the fluid, and the detected wave after the interaction with fluid indicates the flow velocity of the fluid. In one example, the flow velocity measurement can be performed in any suitable way, such as by measuring a Doppler shift.

In some examples, the at least one flow sensor(s) 154 comprises at least one optical flow sensor. The term “optical flow sensor”, as used herein, means a flow sensor based on light detection. In one example, an optical flow sensor can include a beam of light configured to heat the blood, and fluctuations in temperature caused by variation in the flow are detected by a fiber optic sensor. In some examples, an optical flow sensor can include a pair of light beams and a detection mechanism configured to measure the time difference between the scattering of each of the light beams. In some examples, blood flow can be measured through the use of a monochromatic laser diode. For example, the laser probe is inserted into a tissue and turned on, where the light scatters and a small portion is reflected back to the probe. The signal is then processed to calculate the blood flow.

In some examples, the at least one ultrasonic and/or optical flow sensor 154 is directed at the at least one depressed portions 124. The term “directed at”, as used herein, means that a beam output from the at least one ultrasonic and/or optical flow sensor 154 is directed towards the at least one depressed portions 124. In some examples, the at least one ultrasonic and/or optical flow sensor 154 is directed at a predetermined area proximal to the trailing portion 108 of the balloon 100. In some examples, the at least one ultrasonic and/or optical flow sensor 154 is directed at a predetermined area distal to the leading portion 102 of the balloon 100. In some examples, the at least one ultrasonic and/or optical flow sensor 154 is secured to one or more of: the balloon 100; the balloon catheter 20; the nosecone shaft 30; or a dedicated catheter or arm. In some examples, the at least one ultrasonic and/or optical flow sensor 154 is positioned within the cavity 132 of balloon 100 (examples not shown explicitly).

In some examples, the at least one flow sensor(s) 154 comprises at least one mechanical flow sensor. The term “mechanical flow sensor”, as used herein, means a flow sensor configured to detect flow based on mechanical effects of the fluid flow, as known to those skilled in the art. These mechanical effects can include positive-displacement based flowmeters and/or pressure-based flowmeters.

In some examples, the flow sensor(s) 154 are secured at respective positions proximal to the trailing portion 108 of the balloon 100. In some examples, the flow sensor(s) 154 are secured at respective predetermined positions distal to the leading portion 102 of the balloon 100. In some examples, a plurality of flow sensors 154 are positioned proximal to the trailing portion 108 of the balloon 100, each of the plurality of flow sensors 154 positioned proximal to an adjacent one of the plurality of flow sensors 154. Thus, the flow sensors 154 are in such an example sequentially positioned away from the trailing portion 108. Advantageously, this avoids identifying turbulence within the blood as actual flow. Thus, flow can be detected if all of the flow sensors 154 detect the flow. In the event that only one, or some, of the flow sensors 154 detect flow, it will be identified as turbulence or other type of noise.

In some examples, the at least one flow sensor(s) 154 is in mechanical communication with the balloon 100. The term “mechanical communication”, as used herein, means that it is attached to the balloon 100 or attached to an element that is attached to the balloon (directly or indirectly), including examples where the at least one flow sensor(s) 154 is attached to the balloon catheter 20 or the nosecone shaft 30, which in turn are attached to the balloon 100, and including examples where the at least one flow sensor(s) resides within the cavity 132. Thus, in some examples, the at least one flow sensor(s) 154 is attached to the balloon 100, the balloon catheter 20 and/or another element of the delivery apparatus 10.

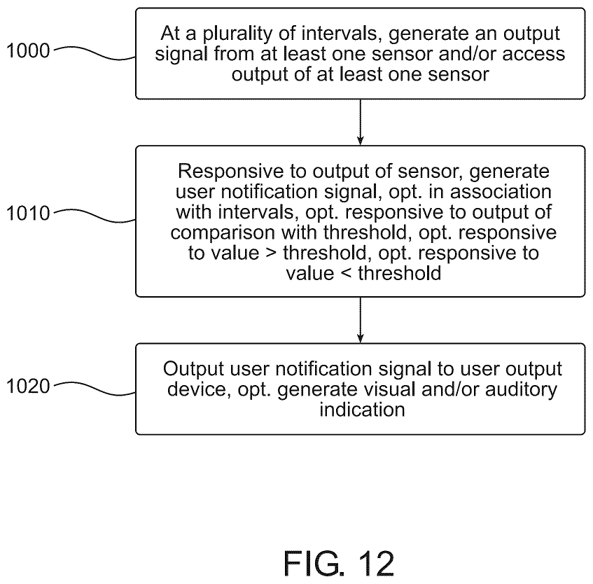

The following is described in relation to the flow chart of FIG. 12. In step 1000, in some examples the at least one sensor 150 is configured to generate an output signal, at an output thereof, at a plurality of predetermined intervals. Alternatively, the at least one sensor 150 is configured to generate the output signal continuously. In some examples, the generated output signal comprises an indication of the measurement of the respective at least one sensor 150. For example, if the at least one sensor 150 comprises a force sensor, the generated output signal comprises an indication (e.g., the voltage value of the signal) of the measured force. Similarly, for example, if the at least one sensor 150 comprises a pressure sensor, the generated output signal comprises an indication of the measured pressure. Similarly, for example, if the at least one sensor 150 comprises a flow sensor 154, the generated output signal comprises an indication of the measured flow.

In some examples, sensor data unit 60 compares the velocity of blood flow, measured by flow sensor(s) 154, to at least one predetermined flow reference value and outputs an output signal based at least in part on an outcome of the comparison. The output signal can comprise an indication of the difference between the flow measurement and the reference value, or another predetermined function thereof. In some examples, sensor data unit 60 compares the velocity of blood flow to a plurality of predetermined flow reference values. Sensor data unit 60 then outputs an output signal which is indicative of the measured velocity of blood flow. For example, the output signal can indicate which of the predetermined reference values the measured blood flow is closest to.

In such an example, user output device 62 can indicate one of a plurality of general flow velocities (e.g., very slow, slow, medium, or standard flow velocity) and a physician/technician monitoring user output device 62 can use this information to adjust the speed of inflation. For example, as will be described below, the faster the flow velocity is, the slower the inflation can be.

In some examples, sensor data unit 60 determines a desired inflation rate, based at least in part on the measured velocity of blood flow. The term “desired inflation rate”, as used herein, means an inflation rate of balloon 100 which is suggested to the physician/technician. In some examples, the desired inflation rate exhibits a predetermined negative correlation with the measured velocity of blood flow. For example, as described above, if the measured velocity of blood flow is slow, the desired inflation rate of balloon 100 will be fast in order to rapidly complete the inflation to avoid any adverse effects to the limited blood flow. In contrast, if the measured velocity of blood flow is faster, the desired inflation rate of balloon 100 can be slower in order to allow more time for accurate deployment. This is possible since blood flow is not as limited. In some examples, the determined desired inflation rate can be output by user output device 62.

In some examples, sensor data unit 60 determines a desired inflation time, based at least in part on the measured velocity of blood flow. The term “desired inflation time”, as used herein, means a time period for inflation of balloon 100 which is suggested to the physician/technician, i.e. an amount of time allowed for inflating balloon 100 before there are adverse effects due to limited blood flow. In some examples, the desired inflation time exhibits a predetermined positive correlation with the measured velocity of blood flow. For example, if the measured velocity of blood flow is slow, the desired inflation time of balloon 100 will be short in order to rapidly complete the inflation to avoid any adverse effects to the limited blood flow. In contrast, if the measured velocity of blood flow is faster, the desired inflation time of balloon 100 can be longer in order to allow more time for accurate deployment. This is possible since blood flow is not as limited. In some examples, the determined desired inflation time can be output by user output device 62.

In some examples, sensor data unit 60 compares the measured velocity of blood flow to one, or both, of: a table comprising predetermined relationships between blood flow velocity values and desired inflation rate/desired inflation time; or a curve representing the predetermined relationships between blood flow velocity values and desired inflation rate/desired inflation time.

The predetermined intervals can be equal to each other or different intervals can be defined for different phases of deployment. In some examples, the plurality of predetermined intervals are defined over a predetermined time period, i.e. the output signals are generated, at predetermined intervals, over the predetermined time period. In some examples, the predetermined time period is about 1 minute. In some examples, the predetermined time period is between 0.5-10 minutes. In some examples, the predetermined time intervals are defined as 0.1-5 seconds. In some examples, the at least one sensor 150 is operated by the sensor data unit 60 (e.g., a voltage and current is provided to the at least one sensor 150 by the sensor data unit 60) and the sensor data unit 60 operates the at least one sensor 150 at the respective predetermined time intervals.

In some examples, the sensor data unit 60 is configured to access the output of the at least one sensor 150 at a plurality of predetermined intervals. In some examples, these are the same predetermined intervals as those of the generated output signals of the at least one sensor 150. Alternatively, the at least one sensor 150 generates output signals at shorter intervals, or continuously, and the sensor data unit 60 accesses the generated output signals at different intervals. For example, the at least one sensor 150 may generated output signals every 10 milliseconds, and the sensor data unit 60 can access these generated output signals every 100 milliseconds.

The term “access”, as used herein, means that the sensor data unit 60 performs a predetermined action with generated output signal of the at least one sensor 150. This can include, without limitation, reading the output signal (and optionally storing it in a memory), applying an analog-to-digital converted to the output signal, comparing the output signal to a predetermined value (in digital and/or analog form), or applying a predetermined function to the value of the output signal (in digital and/or analog form).

In some examples, the at least one sensor 150 generates output signals continuously and/or the sensor data unit 60 accesses these signals continuously.

In step 1010, in some examples the sensor data unit 60 is configured, based at least in part on the output of the at least one sensor 150 (e.g., based at least in part on the generated output signal), to generate a user notification signal. In some examples, in step 1020, the generated user notification signal is transmitted to the user output device 62. In some examples, the user notification signal is any, or a combination of: one or more software instructions sent to the user output device 62; or an analog or digital signal sent to the user output device 62 (via a communication device or wirelessly via an antenna).

In some examples, the user notification signal is generated in associated with each of the plurality of predetermined intervals. The term “associated with each of the plurality of predetermined intervals”, as used herein, means that the time at which the user notification signal is generated has a predetermined relationship with the predetermined intervals. For example, each user notification signal can be generated after the latency time from when the respective output signal of the at least one sensor 150 is accessed. In some examples, the user notification signal is generated at the same predetermined intervals of the access of the output signal of the at least one sensor 150. For example, the user notification signal can comprise an indication of the measurement of the respective at least one sensor 150, as described above in relation to the output signal of the at least one sensor 150.

In some examples, the sensor data unit 60 compares a value of the output of the at least one sensor 150 (e.g., the voltage or amplitude value of the generated output signal) to a predetermined threshold and the user notification signal is generated responsive an outcome of the comparison. For example, the user notification signal can comprise an indication of whether or not the value exceeds, or is less than, the threshold. In some examples, the user notification signal is generated based at least in part on the outcome of the comparison meeting a predetermined condition. The condition can include: generating the user notification signal based at least in part on the outcome of the comparison indicating that the measured value exceeds the threshold value; or generating the user notification signal based at least in part on the outcome of the comparison indicating that the measured value has not yet reached the threshold value.

Thus, for example, the user notification signal can control the user output device 62 to output an indication to the user that the pressure/force has reached the limit and inflation should be stopped. Alternatively, the user notification signal can control the user output device 62 to output an indication to the user that the pressure/force has not yet reached the limit and inflation can continue. Similarly, the user notification signal can control the user output device 62 to output an indication to the user that the flow of blood has stopped, or alternatively that the flow of blood has not yet stopped. In some examples, an indication of both the blood flow and the pressure/force measurements are output since a lack of blood flow can be indicative that the inflatable balloon 100 is fully inflated or that the inflatable balloon 100 is fully deflated and the at least one flow sensor 154 is hidden within creases of the inflatable balloon 100 and therefore cannot detect blood flow.

In some examples, the user output device 62 is configured to generate, based at least in part on the generated user notification signal of sensor data unit 60, a visual and/or auditory indication, as described above. Thus, the above system allows a physician or technician to gradually inflate the inflatable balloon 100, while the user output device 62 indicates whether more inflation is necessary/possible. For example, the shape of the inflatable balloon 100 allows gradual inflation by allowing blood flow through channels 126, thereby providing more time to accurately deploy the prosthetic device 12 without completely blocking blood flow. This provided time, allows the physician/technician to keep track of the results of the inflation from user output device 62, and inflate/deflate the inflatable balloon 100 as necessary. This is in contrast to conventional inflatable balloons that completely block blood flow when inflated, requiring the balloon inflation and prosthetic valve implantation procedures to be performed in a relatively short period of time, in which case the balloon is rapidly inflated such that even when including force or pressure sensors attached thereto, by the time the balloon is inflated and the clinician gets the chance to review the acquired force or pressure measurement, the valve may have already been overexpanded.

In some examples, sensor data unit 60 transmits information regarding the output signals of the at least one sensor 150 to the external system 300. In some examples, the information comprises the measurements of the at least one sensor 150. In some examples, external system 300 analyzes the received information to determine an optimized method of inflating the inflatable balloon 100, optionally using a convolutional neural network or other machine learning algorithms.

In some examples, the sensor data unit 60 stores and/or transmits to the external system 300 any of: the values measured by the at least one sensor 150, such as force values, pressure values and/or flow values; the volume of inflation fluid 35 injected between successive measurements, or over a predetermined time period preceding the respective measurement; the rate of injection of the inflation fluid 35, or in the case of incremental inflation, information regarding the length and/or frequency of each increment; at what point the respective threshold value was reached (e.g., how much inflation fluid 35 was injected before reaching the respective threshold value). Additionally information stored and/or transmitted to the external system 300 can include: patient information, such as age, sex and/or morphological information of the patient lumen derived from previous imaging (e.g., the diameter of the native valve); and/or procedure information, such as type and size of the balloon and/or type and size of the prosthetic valve.

In some examples, the machine learning algorithm, analyzes the patient information and procedure information for a plurality of patients/procedures and determines any of the following, without limitation: optimal type/size of prosthetic valve and/or balloon for respective patient parameters; and/or optimal inflation rate/increment parameters of the balloon (e.g., increment frequency and/or inflation fluid volume for each increment).

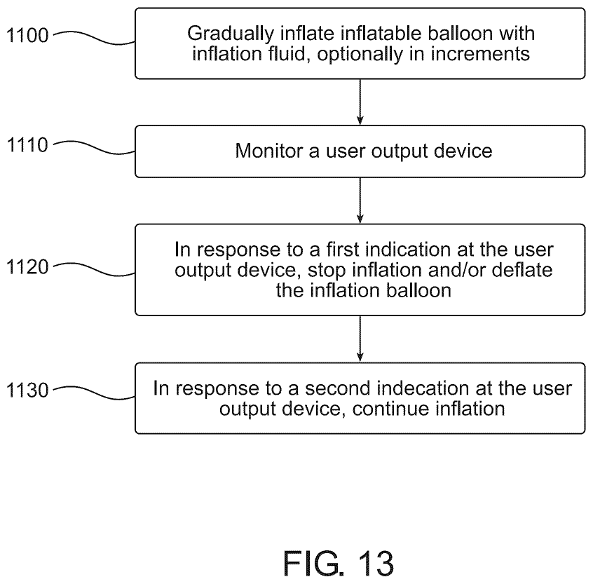

FIG. 13 illustrates a high-level flow chart of a method of delivering an inflatable balloon with a balloon catheter into a patient lumen. In some examples, in step 1100, the inflatable balloon is delivered into the patient lumen and is inflated. For example, an inflation fluid 35 is injected into the inflatable balloon, the injected fluid being what inflates the balloon.

In some examples, the inflatable balloon is gradually inflated. The term “gradually”, as used herein, means that the inflatable balloon is inflated at a slow enough pace such that a physician/technician can stop the inflation before the balloon is completely inflated if necessary. For example, in some examples, the pace of inflation is generally determined such that if a visual or auditory indication is presented to the physician/technician, the physician/technician will be able to react before inflation is completed. In some examples, the rate of inflation is physically limited by the syringe or pump being used to inflate the inflatable balloon. The rate of inflation can also be physically limited at any point along fluid passageway 34.

In some examples, inflation of the inflatable balloon is performed in increments. For example, the physician/technician injects a first portion of the fluid to partially inflate the inflatable balloon, and then proceeds to inject the next portion of the fluid if possible.

In some examples, at a first increment a first volume of the inflation fluid 35 is injected. In some examples, the first volume is a predetermined fraction of the nominal inflation volume of the inflation fluid 35. The term “nominal inflation volume”, as used herein, means a volume of inflation fluid 35 defined by the manufacturer which is recommended for inflation of the respective balloon to a nominal balloon diameter, as known to those skilled in the art. The term “nominal balloon diameter”, as used herein, means a diameter of the balloon defined by the manufacturer to be the preferred diameter of the balloon after completing inflation. As will be described below, the next increment(s) depend on monitoring the output of a user output device.

In some examples, information is received regarding the blood flow across the balloon. As described above, this information can include, without limitation: the presence or absence of blood flow across the balloon; the measured velocity of blood flow across the balloon; an indication of an outcome of comparison of the measured velocity to a predetermined threshold.

In some examples, based at least in part on the received information, injection of the inflation fluid into the balloon is stopped, as described above and further described below. In some examples, a second volume of the inflation fluid is injected into the balloon. In some examples, the second volume is a second fraction of the nominal inflation volume. The second fraction of the nominal inflation volume can be determined before the procedure (e.g. a first volume is defined in advance for a first injection and a second volume is defined in advance for a second injection). In some examples, the second volume of the inflation fluid is determined based at least in part on the received information. In some examples, the second volume can be determined based on the difference between the measured blood flow velocity and a respective predetermined threshold.

In step 1110, in some examples, a user output device is monitored by the physician/technician, such as user output device 62 described above. In one example, the user output device is monitored to detect the presence of blood flow across the balloon. In some examples, the physician/technician monitors the user output device to determine whether the inflatable balloon can, or should, be further inflated. For example, the user output device can indicate whether the pressure or force applied by the inflatable balloon is greater than the maximum threshold value. In some examples, during inflation the tissue of the patient lumen is stretched. Gradually, the tissue relaxes, thereby allowing additional inflation of the inflatable balloon. In some examples, the physician/technician can stop inflating the inflatable balloon for a predetermined amount of time and monitor the user output device. For example, the inflation can be stopped for at least 2 seconds, optionally at least 5 second, and further optionally at least 10 seconds. In some examples, the physician/technician can stop inflating the inflatable balloon until there is an indication by the user output device that measurements (e.g., force/pressure values) decrease by a predetermined amount, decrease to a predetermined value and/or decrease at a predetermined rate (e.g., there may be a rapid decrease in pressure initially, and then the decrease slows as the tissue approaches full relaxation). In some examples, the physician/technician can stop the inflation for the predetermined time period or until the respective indication is output, whichever occurs first.

In step 1120, in some examples, in response to a first indication at the user output device, the physician/technician stops further inflation of the inflatable balloon. For example, the first indication can be an indication that the maximum pressure applied by the balloon has been reached or that blood flow has dropped to below a respective threshold value. In some examples, the physician/technician can partially deflate the inflatable balloon by removing some of the fluid. As described above, the physician/technician can stop inflation at predetermined intervals.

In some examples, additional volume of the inflation fluid 35 is injected until a first predetermined condition is satisfied. In some examples, the first predetermined condition is associated with the measured velocity of the blood flow across the balloon and/or the measured force value between the balloon and the prosthetic device. In some examples, the first predetermined condition is a graphical display of any of: the blood flow velocity across the balloon; an indication of the presence or absence of blood flow across the balloon. It is noted that this list is not limiting and any other data can be displayed on a graphical display without exceeding the scope of the disclosure.

In some examples, the first predetermined condition includes a respective outcome of a comparison of information associated with blood flow across the balloon to a predetermined value, such as the measured blood flow velocity being less than a respective predetermined threshold.

In some examples, additional volume of the inflation fluid 35 is injected until a second predetermined condition is satisfied. In some examples, the second predetermined condition is associated with the measurement of the force and/or pressure applied between the balloon and the prosthetic valve and/or the patient lumen, as described above. In some examples, the second predetermined condition is a graphical display of a measurement of a force and/or pressure applied between the balloon and the prosthetic valve. In some examples, the second predetermined condition includes a respective outcome of a comparison of information associated with the measured force/pressure to a respective predetermined value, such as the measured force/pressure being greater than a respective predetermined threshold.

In step 1130, in some examples, in response to a second indication at the user output device, the physician/technician continues to inflate the inflatable balloon by injecting addition fluid, as described above. For example, the second indication can be an indication that the maximum pressure applied by the balloon has not been reached, thus the balloon can be further inflated.

While utilization of sensors 150, such as utilization of flow sensors 154, with or without force sensors 152, are described above and illustrated for use in combination with perfusion balloons 100 that form channels 126 extending along their central portions, optionally in a helical configuration, it is to be understood that any method and configuration described for utilizing flow sensor 154, with or without other sensors, described herein, can be similarly utilized with other types or shapes of inflatable balloons configured to permit blood flow therealong or therethrough when in an inflated state. For example, some types of inflatable balloons are disclosed in U.S. Pat. No. 9,707,078, which is incorporated herein by reference.

In some examples, flow sensor(s) 154 can be utilized according to any of the above mentioned methods and configurations, including detecting the presence of blood flow, and optionally measuring such flow, along balloon assemblies that include a plurality of outer balloon members disposed around an inner balloon member, or an outer balloon member helically wrapped around an inner balloon member, configured to form voids in an inflated state thereof to allow blood perfusion therethrough.

In some examples, flow sensor(s) 154 can be utilized according to any of the above mentioned methods and configurations, including detecting the presence of blood flow, and optionally measuring such flow, along a balloon provided in the form of elongated or tubular balloon helically wound around the longitudinal axis of the delivery apparatus.

In some examples, flow sensor(s) 154 can be utilized according to any of the above mentioned methods and configurations, including detecting the presence of blood flow, and optionally measuring such flow, along a balloon that includes outer constriction members, such as helical wires or mold bands disposed around the outer surface of the balloon, configured to restrict balloon expansion to the portions disposed between such members.

In some examples, flow sensor(s) 154 can be utilized according to any of the above mentioned methods and configurations, including detecting the presence of blood flow, and optionally measuring such flow, along a balloon that includes internal biasing means, such as a spring coil disposed within the balloon's cavity, configured to transition from an elongated compacted configuration during delivery, to an outwardly biased configuration pushing against the balloon wall to force it into the helical configuration.

In some examples, flow sensor(s) 154 can be utilized according to any of the above mentioned methods and configurations, including detecting the presence of blood flow, and optionally measuring such flow, along a balloon configured to have an outer surface of a portion contact an outer surface of an internal shaft, such as a nosecone shaft, which serves to form a flow channel around the balloon in its inflated state.

In some examples, flow sensor(s) 154 can be utilized according to any of the above mentioned methods and configurations, including detecting the presence of blood flow, and optionally measuring such flow, along a balloon that includes a plurality of projections extending radially outward from a main body thereof, wherein the projections define grooves in-between configured to permit blood flow between both ends of the balloon.

In some examples, flow sensor(s) 154 can be utilized according to any of the above mentioned methods and configurations, including detecting the presence of blood flow, and optionally measuring such flow, along a balloon or balloon assembly that includes one or more toroid or donut shaped balloon(s), configured to permit blood flow through a central perfusion lumen defined thereby.

In some examples, as described above, information regarding the presence of blood flow across the balloon is received. As described above, the information can include an indication whether or not blood flow is present across the balloon, a measurement of the blood flow across the balloon and/or an outcome of a comparison of the measured blood flow to one or more values.

### Some Examples of the Disclosed Technology

Some examples of the above-described technology are enumerated below. It should be noted that one feature of an example in isolation or more than one feature of the example taken in combination and, optionally, in combination with one or more features of one or more examples below are examples also falling within the disclosure of this application.

Example 1. A delivery apparatus, comprising:
a balloon movable between a deflated state and an inflated state, the balloon comprising: an internal cavity;
a central portion disposed around the cavity and extending along a length between a leading portion and a trailing portion of the balloon, wherein the central portion comprises at least two lobes spiraling around a longitudinal axis of the balloon and along the length of the central portion, and at least two depressed portions extending between the lobes; and
a balloon catheter coupled to the balloon, wherein the balloon catheter comprises a balloon catheter lumen in fluid communication with the cavity of the balloon via one or more openings of the balloon catheter;
wherein, when the balloon is in the inflated state within a target lumen, at least two channels are provided between the depressed portions and an inner surface of the target lumen to allow blood to flow through the channels and across the balloon.

Example 2. The delivery apparatus of any example herein, particularly example 1, wherein the lobes and the depressed portions together form a cross-sectional contour devoid of acute or right angles between the lobes and the depressed portions.

Example 3. The delivery apparatus of any example herein, particularly example 1 or 2, wherein the balloon defines, in the inflated state, a major radial distance between the longitudinal axis and any of the lobes, and a minor radial distance between the longitudinal axis and any of the depressed portions, wherein the major radial distance is greater than the minor radial distance.

Example 4. The delivery apparatus of any example herein, particularly example 3, wherein the major radial distance is identical for all of the lobes, and wherein the minor radial distance is identical for all of the depressed portions.

Example 5. The delivery apparatus any example herein, particularly any one of examples 1 to 4, wherein the balloon is a unitary balloon and wherein the internal cavity is a single internal cavity.

Example 6. The delivery apparatus any example herein, particularly any one of examples 1 to 5, wherein the balloon is not wound around any other shaft or catheter extending along the longitudinal axis.

Example 7. The delivery apparatus any example herein, particularly any one of examples 1 to 6, wherein the delivery apparatus is devoid of constriction elements helically disposed around the balloon.

Example 8. The delivery apparatus any example herein, particularly any one of examples 1 to 7, wherein the delivery apparatus is devoid of biasing means disposed in the cavity, configured to press against the balloon in the inflated state to force it into a helically-shaped configuration.

Example 9. The delivery apparatus any example herein, particularly any one of examples 1 to 8, wherein no portion of an outer surface of the central portion of the balloon may contact an outer surface of any shaft or catheter extending through the cavity, in the inflated state.

Example 10. The delivery apparatus any example herein, particularly any one of examples 1 to 9, wherein the at least two lobes and the at least two depressed portions together define a continuous surface of the balloon.

Example 11. The delivery apparatus any example herein, particularly any one of examples 1 to 10, wherein a curvature exhibited by any of the lobes is greater than a curvature exhibited by any of the depressed portions.

Example 12. The delivery apparatus of any example herein, particularly example 11, wherein the curvatures of all of the lobes are identical, and wherein the curvatures of all of the depressed portions are identical.

Example 13. The delivery apparatus any example herein, particularly any one of examples 1 to 12, wherein the cross-sectional shape of the balloon across any plane which is orthogonal to the longitudinal axis, along the length of the central portion, is identically shaped but angularly offset with respect to the cross-sectional shape across a preceding or a succeeding orthogonal plane.

Example 14. The delivery apparatus any example herein, particularly any one of examples 1 to 13, wherein the lobes are parallel to each other, and wherein the depressed portions are parallel to each other.

Example 15. The delivery apparatus any example herein, particularly any one of examples 1 to 14, wherein the at least two lobes comprise three lobes, and wherein the at least two depressed portions comprise three depressed portions.

Example 16. The delivery apparatus any example herein, particularly any one of examples 1 to 15, wherein the leading and the trailing portions taper, in the inflated state, from the respective ends of the central portion to narrower diameters at their opposite ends.

Example 17. The delivery apparatus any example herein, particularly any one of examples 1 to 15, wherein leading and the trailing portions have non-circular cross-sectional shapes along at least a portion of tapering segments thereof in the inflated state.

Example 18. The delivery apparatus any example herein, particularly any one of examples 1 to 17, further comprising a handle through which the balloon catheter extends.

Example 19. The delivery apparatus any example herein, particularly any one of examples 1 to 18, wherein the balloon catheter further comprises a proximal portion having a fluid passageway which is in fluid communication with the balloon catheter lumen.

Example 20. The delivery apparatus any example herein, particularly any one of examples 1 to 19, further comprising a delivery shaft disposed around at least a portion of the balloon catheter, wherein the delivery shaft and the balloon catheter are configured to slide longitudinally relative to each other.

Example 21. The delivery apparatus any example herein, particularly any one of examples 1 to 20, further comprising a nosecone mounted on a distal end of the delivery apparatus.

Example 22. The delivery apparatus of any example herein, particularly example 21, further comprising a nosecone shaft attached to the nosecone and extending though the balloon catheter, wherein the balloon catheter and the nosecone shaft are configured to slide longitudinally relative to each other.

Example 23. The delivery apparatus any example herein, particularly any one of examples 1 to 22, wherein the trailing portion is attached to the balloon catheter.

Example 24. The delivery apparatus any example herein, particularly any one of examples 1 to 23, wherein the leading portion is attached to the balloon catheter.

Example 25. The delivery apparatus any example herein, particularly example 21, wherein the leading portion is attached to the nosecone.

Example 26. The delivery apparatus any example herein, particularly any one of examples 1 to 25, wherein each lobe exhibits a curved cross-sectional convex shape, and wherein each depressed portion exhibits a curved cross-sectional concave shape.

Example 27. The delivery apparatus any example herein, particularly any one of examples 1 to 25, wherein each lobe exhibits a curved cross-sectional convex shape, and wherein each depressed portion exhibits a cross-sectional linear shape along at least a portion thereof.

Example 28. The delivery apparatus any example herein, particularly any one of examples 1 to 26, wherein the lobes and the depressed portions exhibit cross-sectional curved shapes devoid of sharp edges.

Example 29. The delivery apparatus any example herein, particularly any one of examples 1 to 28, further comprising a prosthetic device disposed over the balloon catheter or the balloon.

Example 30. The delivery apparatus of any example herein, particularly example 29, wherein the lobes are configured to press against the prosthetic device when the balloon is inflated to expand the prosthetic device.

Example 31. The delivery apparatus of any example herein, particularly example 30, wherein the depressed portions are distanced from the prosthetic valve in the inflated state.

Example 32. The delivery apparatus any example herein, particularly any one of examples 29 to 31, wherein the prosthetic device is a prosthetic valve comprising an annular frame and a valvular structure mounted within the frame, the valvular structure comprising a plurality of leaflets coupled to each other and to the frame via a plurality of commissures.

Example 33. The delivery apparatus any example herein, particularly any one of examples 1 to 32, wherein the cross-sectional shape of the central portion is non-circular.

Example 34. The delivery apparatus any example herein, particularly any one of examples 1 to 33, wherein the central portion has a uniform cross-sectional area along its entire length.

Example 35. The delivery apparatus any example herein, particularly any one of examples 1 to 34, further comprising at least one flow sensor configured to detect the presence of blood flow across the balloon.

Example 36. The delivery apparatus of any example herein, particularly example 35, wherein the at least one flow sensor is configured to measure the velocity of blood flow across the balloon.

Example 37. The delivery apparatus of any example herein, particularly example 35 or 36, wherein the at least one flow sensor is attached to the balloon, the balloon catheter and/or another portion of the delivery apparatus.

Example 38. The delivery apparatus of any example herein, particularly any one of examples 35 to 37, wherein the at least one flow sensor is attached to the balloon.

Example 39. The delivery apparatus of any example herein, particularly example 38, wherein the at least one flow sensor is attached to at least one of the depressed portions.

Example 40. The delivery apparatus of any example herein, particularly example 39, wherein the at least one flow sensor comprises at least one flow sensor attached to each of the depressed portions.

Example 41. The delivery apparatus of any example herein, particularly any one of examples 35 to 40, wherein the at least one flow sensor comprises at least one ultrasonic and/or optical flow sensor.

Example 42. The delivery apparatus of any example herein, particularly example 41, wherein the at least one ultrasonic and/or optical flow sensor is directed at one or more of the channels.

Example 43. The delivery apparatus of any example herein, particularly any one of examples 35 to 42, wherein the at least one ultrasonic flow sensor is directed at a predetermined area proximal to the trailing portion of the balloon and/or at a predetermined area distal to the leading portion of the balloon.

Example 44. The delivery apparatus of any example herein, particularly any one of examples 35 to 43, wherein the at least one flow sensor is secured at a predetermined position proximal to the trailing portion of the balloon and/or at predetermined position distal to the leading portion of the balloon.

Example 45. The delivery apparatus of any example herein, particularly example 44, wherein the at least one flow sensor comprises a plurality of flow sensors positioned proximal to the trailing portion of the balloon, each of the plurality of flow sensors positioned proximal to an adjacent one of the plurality of flow sensors.

Example 46. The delivery apparatus of any example herein, particularly example 36, further comprising a sensor data unit and at least one communication device, wherein the output of the at least one sensor is in communication with the sensor data unit via the at least one communication device, wherein the sensor data unit is configured to compare the measured velocity of blood flow to at least one predetermined flow reference value and output an output signal based at least in part on an outcome of the comparison.

Example 47. The delivery apparatus of any example herein, particularly example 46, wherein the at least one predetermined flow reference value comprises a plurality of predetermined flow reference values and the output signal is indicative of the measured velocity of blood flow.

Example 48. The delivery apparatus of any example herein, particularly example 46 or 47, wherein the sensor data unit is configured, based at least in part on the measured velocity of blood flow, to determine a desired inflation rate, the desired inflation rate exhibiting a predetermined negative correlation with the measured velocity of blood flow, wherein the output signal is indicative of the desired inflation rate.

Example 49. The delivery apparatus of any example herein, particularly example 46 or 47, wherein the sensor data unit is configured, based at least in part on the measured velocity of blood flow, to determine a desired inflation time, the desired inflation time period exhibiting a predetermined positive correlation with the measured velocity of blood flow, wherein the output signal is indicative of the desired inflation time period.

Example 50. The delivery apparatus any example herein, particularly any one of examples 1 to 49, further comprising at least one force sensor.

Example 51. The delivery apparatus of any example herein, particularly example 50, wherein the at least one force sensor is attached to a lobe outer surface of at least one of the lobes.

Example 52. The delivery apparatus of any example herein, particularly example 51, wherein the lobe outer surface defines a width that is at least as great as the width of the force sensor.

Example 53. The delivery apparatus of any example herein, particularly example 52, wherein the lobe outer surface remains uncurved along its width during the deflated and inflated states of the balloon.

Example 54. The delivery apparatus any example herein, particularly any one of examples 50 to 53, wherein the at least one force sensor comprises at least one force sensor attached to each of the lobes.

Example 55. The delivery apparatus any example herein, particularly any one of examples 50 to 54, further comprising a sensor data unit and at least one communication device, wherein the at least one sensor is in communication with the sensor data unit via the at least one communication device, wherein the at least one force sensor comprises a plurality of force sensors; and wherein the sensor data unit is configured, based at least in part on an output of the plurality of force sensors, to generate a map of forces applied to the balloon.

Example 56. The delivery apparatus of any example herein, particularly example 55, wherein the sensor data unit is further configured to output an indication of the determined map.

Example 57. The delivery apparatus of any example herein, particularly example 56, wherein the sensor data unit is further configured, based at least in part on the determined map of forces, to: determine whether implantation of a balloon expandable prosthetic valve at an anatomical location of the balloon is viable, and output an indication of the determined viability.

Example 58. A delivery apparatus, comprising:
a balloon movable between a deflated state and an inflated state, the balloon comprising: an internal cavity;
a central portion disposed around the cavity and extending along a length between a leading portion and a trailing portion of the balloon, wherein the central portion exhibits, at least in the inflated state: at least two lobes spiraling around a longitudinal axis of the balloon and along the length of the central portion, and at least two depressed portions extending between the lobes;
a balloon catheter coupled to the balloon, wherein the balloon catheter comprises a balloon catheter lumen which is exposed to the cavity of the balloon via one or more openings of the balloon catheter; and
at least one sensor;
wherein, when the balloon is in the inflated state within a target lumen and/or a prosthetic device, at least two channels are provided between the depressed portions and an inner surface of the target lumen and/or prosthetic device to allow blood to flow through the channels and across the balloon, and
wherein the at least one sensor comprises at least one flow sensor configured to detect the presence of blood flow across the balloon.

Example 59. The delivery apparatus of any example herein, particularly example 58, wherein the at least one flow sensor is configured to measure the velocity of blood flow across the balloon.

Example 60. The delivery apparatus of any example herein, particularly 58 or 59, wherein the at least one flow sensor is attached to the balloon, the balloon catheter and/or another portion of the delivery apparatus.

Example 61. The delivery apparatus of any example herein, particularly any one of examples 58 to 60, wherein the at least one flow sensor is attached to the balloon.

Example 62. The delivery apparatus of any example herein, particularly example 61, wherein the at least one flow sensor is attached to at least one of the depressed portions.

Example 63. The delivery apparatus of any example herein, particularly example 62, wherein the at least one flow sensor comprises at least one flow sensor attached to each of the depressed portions.

Example 64. The delivery apparatus of any example herein, particularly any one of examples 58 to 63, wherein the at least one flow sensor comprises at least one ultrasonic and/or optical flow sensor.

Example 65. The delivery apparatus of any example herein, particularly example 64, wherein the at least one ultrasonic and/or optical flow sensor is directed at one or more of the channels.

Example 66. The delivery apparatus of any example herein, particularly any one of examples 58 to 64, wherein the at least one ultrasonic and/or optical flow sensor is directed at a predetermined area proximal to the trailing portion of the balloon and/or at a predetermined area distal to the leading portion of the balloon.

Example 67. The delivery apparatus of any example herein, particularly any one of examples 58 to 66, wherein the at least one flow sensor is secured at a predetermined position proximal to the trailing portion of the balloon and/or at predetermined position distal to the leading portion of the balloon.

Example 68. The delivery apparatus of any example herein, particularly example 67, wherein the at least one flow sensor comprises a plurality of flow sensors positioned proximal to the trailing portion of the balloon, each of the plurality of flow sensors positioned proximal to an adjacent one of the plurality of flow sensors.

Example 69. The delivery apparatus of any example herein, particularly any one of examples 58 to 68, wherein the at least one sensor further comprises at least one force sensor.

Example 70. The delivery apparatus of any example herein, particularly example 69, wherein the at least one force sensor is attached to a lobe outer surface of at least one of the lobes.

Example 71. The delivery apparatus of any example herein, particularly example 70, wherein the lobe outer surface defines a width that is at least as great as the width of the force sensor.

Example 72. The delivery apparatus of any example herein, particularly any one of examples 69 to 71, wherein the at least one force sensor comprises at least one force sensor attached to each of the lobes.

Example 73. The delivery apparatus of any example herein, particularly any one of examples 58 to 72, further comprising a sensor data unit and at least one communication device, wherein the output of the at least one sensor is in communication with the sensor data unit via the at least one communication device.

Example 74. The delivery apparatus of any example herein, particularly example 59, further comprising a sensor data unit and at least one communication device, wherein the output of the at least one sensor is in communication with the sensor data unit via the at least one communication device, and wherein the sensor data unit is configured to compare the measured velocity of blood flow to at least one predetermined flow reference value and output an output signal based at least in part on an outcome of the comparison.

Example 75. The delivery apparatus of any example herein, particularly example 74, wherein the at least one predetermined flow reference value comprises a plurality of predetermined flow reference values and the output signal is indicative of the measured velocity of blood flow.

Example 76. The delivery apparatus of any example herein, particularly example 74 or 75, wherein the sensor data unit is configured, based at least in part on the measured velocity of blood flow, to determine a desired inflation rate, the desired inflation rate negatively correlated with the measured velocity of blood flow, wherein the output signal is indicative of the desired inflation rate.

Example 77. The delivery apparatus of any example herein, particularly example 74 or 75, wherein the sensor data unit is configured, based at least in part on the measured velocity of blood flow, to determine a desired inflation time, the desired inflation time period positively correlated with the measured velocity of blood flow, wherein the output signal is indicative of the desired inflation time period.

Example 78. The delivery apparatus of any example herein, particularly any one of examples 74 to 77, further comprising a user output device configured to generate, based at least in part on the output signal, a visual and/or auditory indication.

Example 79. The delivery apparatus of any example herein, particularly any one of examples 69 to 72, further comprising a sensor data unit and at least one communication device, wherein the output of the at least one sensor is in communication with the sensor data unit via the at least one communication device, wherein the at least one force sensor comprises a plurality of force sensors, and wherein the sensor data unit is configured, based at least in part on the output of the plurality of force sensors, to generate a map of forces applied to the balloon.

Example 80. The delivery apparatus of any example herein, particularly example 79, wherein the sensor data unit is further configured to output an indication of the determined map.

Example 81. The delivery apparatus of any example herein, particularly example 79 or 80, wherein the sensor data unit is further configured, based at least in part on the determined map of forces, to: determine whether implantation of a balloon expandable prosthetic valve at an anatomical location of the balloon is viable, and output an indication of the determined viability.

Example 82. The delivery apparatus of any example herein, particularly any one of examples 73 to 81, further comprising an external system, the sensor data unit in communication with the external system and configured to transmit to the external system information regarding the output of the at least one sensor.

Example 83. A method comprising delivering a balloon in a deflated state thereof, mounted on a balloon catheter, through the vasculature of the patient to a treatment site within a target lumen, wherein the balloon comprises an internal cavity and a central portion disposed around the cavity and extending along a length between a leading portion and a trailing portion of the balloon, wherein the central portion comprises at least two lobes spiraling around a longitudinal axis of the balloon and along the length of the central portion, and at least two depressed portions extending between the lobes,
wherein the method further comprises, based at least in part on an output of at least one sensor, inflating the balloon within the target lumen,
wherein, when the balloon is in an inflated state, at least two channels are provided between the depressed portions and an inner surface of the target lumen to allow blood to flow through the channels and across the balloon, and
wherein the at least one sensor comprises at least one flow sensor configured to detect the presence of blood flow across the balloon.

Example 84. The method of any example herein, particularly example 83, further comprising: measuring, by the at least one sensor, the velocity of blood flow across the balloon; comparing the measured velocity of blood flow to at least one predetermined flow reference value; and determining a desired inflation rate, the desired inflation rate negatively correlated with the measured velocity of blood flow, wherein the rate of inflation of the balloon is based at least in part on the determined desired inflation rate.

Example 85. The method of any example herein, particularly example 83, further comprising: measuring, by the at least one sensor, the velocity of blood flow across the balloon; comparing the measured velocity of blood flow to at least one predetermined flow reference value; and determining a desired inflation time period, the desired inflation time period positively correlated with the measured velocity of blood flow, wherein the inflation of the balloon is ceased when the determined desired inflation time period ends.

Example 86. A method comprising: delivering a balloon in a deflated state thereof, mounted on a balloon catheter, through the vasculature of a patient to a treatment site, the balloon comprising an internal cavity and a central portion disposed around the cavity; and inflating the balloon within a target lumen such that the central portion comprises at least two lobes spiraling around a longitudinal axis of the balloon and along the length of the central portion, and at least two depressed portions extending between the lobes, wherein the inflation of the balloon permits blood to pass through at least two channels formed between the depressed portions and the target lumen.

Example 87. The method of any example herein, particularly example 86, wherein inflating the balloon to permit blood to flow through the channels is performed without rapid pacing.

Example 88. The method of any example herein, particularly example 86 or 87, wherein inflating the balloon is performed such that lobe outer surfaces of the lobes press against an inner surface of the target lumen, and wherein the depressed portions are distanced from the inner surface of the target lumen when the balloon is fully inflated against the target lumen.

Example 89. The method of any example herein, particularly example 86 or 87, wherein inflating the balloon is performed such that lobe outer surfaces of the lobes press against an inner surface of a prosthetic device disposed around the balloon, thereby expanding the prosthetic device against an inner surface of the target lumen, and wherein the depressed portions are distanced from the inner surface of the prosthetic device when the balloon is fully inflated against the prosthetic device.

Example 90. The method of any example herein, particularly example 89, wherein the prosthetic device is a prosthetic valve comprising an annular frame and a valvular structure mounted within the frame, the valvular structure comprising a plurality of leaflets coupled to each other and to the frame via a plurality of commissures.

Example 91. The method of any example herein, particularly example 90, wherein the target lumen is an annulus of a native heart valve.

Example 92. The method of any example herein, particularly any one of examples 89 to 91, wherein the prosthetic device is crimped over the balloon when the balloon is delivered to the treatment site.

Example 93. The method of any example herein, particularly any one of examples 89 to 91, wherein the prosthetic device is crimped over a portion of the balloon catheter proximal to the balloon when the balloon is delivered to the treatment site.

Example 94. The method of any example herein, particularly example 93, further comprising advancing the prosthetic device onto the balloon prior to inflating the balloon.

Example 95. The method of any example herein, particularly any one of examples 86 to 94, wherein the lobes and the depressed portions exhibit cross-sectional curved shapes that do not form acute or sharp angles therebetween.

Example 96. The method of any example herein, particularly any one of examples 86 to 95, wherein, when the balloon is inflated, it defines a major radial distance between the longitudinal axis and any of the lobes, and a minor radial distance between the longitudinal axis and any of the depressed portions, wherein the major radial distance is greater than the minor radial distance.

Example 97. The method of any example herein, particularly example 96, wherein the major radial distance is identical for all of the lobes, and wherein the minor radial distance is identical for all of the depressed portions.

Example 98. The method of any example herein, particularly any one of examples 86 to 97, wherein the balloon is a unitary balloon and wherein the internal cavity is a single internal cavity.

Example 99. The method of any example herein, particularly any one of examples 86 to 98, wherein the at least two lobes and the at least two depressed portions together define a continuous surface of the balloon.

Example 100. The method of any example herein, particularly any one of examples 86 to 99, wherein a curvature exhibited by any of the lobes is greater than a curvature exhibited by any of the depressed portions.

Example 101. The method of any example herein, particularly example 100, wherein the curvatures of all of the lobes are identical, and wherein the curvatures of all of the depressed portions are identical.

Example 102. The method of any example herein, particularly any one of examples 86 to 101, wherein the cross-sectional shape of the balloon across any plane which is orthogonal to the longitudinal axis, along the length of the central portion, is identically shaped but angularly offset with respect to the cross-sectional shape across a preceding or a succeeding orthogonal plane.

Example 103. The method of any example herein, particularly any one of examples 86 to 102, wherein the lobes are parallel to each other, and wherein the depressed portions are parallel to each other.

Example 104. The method of any example herein, particularly any one of examples 86 to 103, wherein the at least two lobes comprise three lobes, and wherein the at least two depressed portions comprise three depressed portions.

Example 105. The method of any example herein, particularly any one of examples 86 to 103, wherein the central portion extends between leading and trailing portions of the balloon, and wherein leading and the trailing portions have non-circular cross-sectional shapes along at least a portion of tapering segments thereof after inflating the balloon.

Example 106. The method of any example herein, particularly any one of examples 86 to 105, wherein each lobe exhibits a curved cross-sectional convex shape, and wherein each depressed portion exhibits a curved cross-sectional concave shape.

Example 107. The method of any example herein, particularly any one of examples 86 to 105, wherein each lobe exhibits a curved cross-sectional convex shape, and wherein each depressed portion exhibits a cross-sectional linear shape along at least a portion thereof.

Example 108. The method of any example herein, particularly any one of examples 86 to 106, wherein the lobes and the depressed portions exhibit cross-sectional curved shapes devoid of sharp edges.

Example 109. The method of any example herein, particularly any one of examples 86 to 108, wherein the cross-sectional shape of the central portion is non-circular.

Example 110. The method of any example herein, particularly any one of examples 86 to 109, wherein the central portion has a uniform cross-sectional area along its entire length.

Example 111. The method of any example herein, particularly any one of examples 86 to 110, wherein at least one flow sensor is configured to detect the presence of blood flow across the balloon.

Example 112. The method of any example herein, particularly example 111, wherein the at least one flow sensor is configured to measure the velocity of blood flow across the balloon.

Example 113. The method of any example herein, particularly example 111 or 112, wherein the at least one flow sensor is attached to the balloon, the balloon catheter and/or another portion of the delivery apparatus.

Example 114. The method of any example herein, particularly any one of examples 111 to 113, wherein the at least one flow sensor is attached to the balloon.

Example 115. The method of any example herein, particularly example 114, wherein the at least one flow sensor is attached to at least one of the depressed portions.

Example 116. The method of any example herein, particularly example 115, wherein the at least one flow sensor comprises at least one flow sensor attached to each of the depressed portions.

Example 117. The method of any example herein, particularly any one of examples 111 to 116, wherein the at least one flow sensor comprises at least one ultrasonic and/or optical flow sensor.

Example 118. The method of any example herein, particularly example 117, wherein the at least one ultrasonic and/or optical flow sensor is directed at the at least one depressed portions.

Example 119. The method of any example herein, particularly any one of examples 111 to 118, wherein the central portion extends along a length between a leading portion and a trailing portion of the balloon, and wherein the at least one ultrasonic and/or optical flow sensor is directed at a predetermined area proximal to a trailing portion of the balloon and/or at a predetermined area distal to a leading portion of the balloon.

Example 120. The method of any example herein, particularly any one of examples 111 to 118, wherein the central portion extends along a length between a leading portion and a trailing portion of the balloon, and wherein the at least one flow sensor is secured at a predetermined position proximal to a trailing portion of the balloon and/or at predetermined position distal to a leading portion of the balloon.

Example 121. The method of any example herein, particularly example 120, wherein the at least one flow sensor comprises a plurality of flow sensors positioned proximal to the trailing portion of the balloon, each of the plurality of flow sensors positioned proximal to an adjacent one of the plurality of flow sensors.

Example 122. The method of any example herein, particularly example 121, further comprising comparing the measured velocity of blood flow to at least one predetermined flow reference value and outputting an output signal based at least in part on an outcome of the comparison.

Example 123. The method of any example herein, particularly example 122, wherein the at least one predetermined flow reference value comprises a plurality of predetermined flow reference values and the output signal is indicative of the measured velocity of blood flow.

Example 124. The method of any example herein, particularly example 122 or 123, further comprising, based at least in part on the measured velocity of blood flow, determining a desired inflation rate, the desired inflation rate negatively correlated with the measured velocity of blood flow, wherein the output signal is indicative of the desired inflation rate.

Example 125. The method of any example herein, particularly example 122 or 123, further comprising, based at least in part on the measured velocity of blood flow, determining a desired inflation time, the desired inflation time period positively correlated with the measured velocity of blood flow, wherein the output signal is indicative of the desired inflation time period.

Example 126. The method of any example herein, particularly any one of examples 86 to 125, wherein at least one force sensor is attached to a lobe outer surface of at least one of the lobes.

Example 127. The method of any example herein, particularly example 126, wherein the lobe outer surface defines a width that is at least as great as the width of the force sensor.

Example 128. The method of any example herein, particularly example 127, wherein the lobe outer surface remains uncurved along its width during the deflated and inflated states of the balloon.

Example 129. The method of any example herein, particularly any one of examples 126 to 128, wherein the at least one force sensor comprises at least one force sensor attached to each of the lobes.

Example 130. The method of any example herein, particularly any one of examples 126 to 129, wherein the at least one force sensor comprises a plurality of force sensors, and wherein the method further comprises, based at least in part on an output of the plurality of force sensors, generating a map of forces applied to the balloon.

Example 131. The method of any example herein, particularly example 130, further comprising outputting an indication of the determined map.

Example 132. The method of any example herein, particularly example 131, further comprising, based at least in part on the determined map of forces: determining whether implantation of a balloon expandable prosthetic valve at an anatomical location of the balloon is viable; and outputting an indication of the determined viability.

Example 133. A method comprising: delivering a balloon in a deflated state thereof, mounted on a balloon catheter, through the vasculature of a patient to a treatment site, the balloon comprising an internal cavity and a central portion disposed around the cavity; injecting inflation fluid into the internal cavity of the balloon; receiving information regarding the presence of blood flow across the balloon; and based at least in part on the received information, stopping the injection of the inflation fluid into the balloon, wherein the injection of the inflation fluid into the internal cavity of the balloon inflates the balloon within a target lumen such that the central portion comprises at least two lobes spiraling around a longitudinal axis of the balloon and along the length of the central portion, and at least two depressed portions extending between the lobes, and wherein blood is permitted to pass through at least two channels formed between the depressed portions and the target lumen.

Example 134. The method of any example herein, particularly example 133, wherein the injection of the inflation fluid comprises: injecting a first volume of the inflation fluid into the balloon; and injecting a second volume of the inflation fluid into the balloon, wherein the second volume of the inflation fluid is determined based at least in part on the received information.

Example 135. The method of any example herein, particularly example 134, wherein the first volume of the inflation fluid is a first predetermined fraction of a nominal inflation volume.

Example 136. The method of any example herein, particularly example 134 or 135, further comprising injecting an additional volume of the inflation fluid into the internal cavity of the balloon until a first predetermined condition is satisfied.

Example 137. The method of any example herein, particularly example 136, further comprising measuring an amount of force between the balloon and tissue of the patient, or between the balloon and a prosthetic device, wherein the first predetermined condition is associated with the measured amount of force.

Example 138. The method of any example herein, particularly example 137, further comprising injecting an additional volume of the inflation fluid into the internal cavity of the balloon until a second predetermined condition is satisfied.

Example 139. The method of any example herein, particularly example 138, further comprising measuring an amount of force between the balloon and tissue of the patient, or between the balloon and a prosthetic device, wherein the second predetermined condition is associated with the measured amount of force.

Example 140. The method of any example herein, particularly any one of examples 133 to 139, further comprising measuring the velocity of blood flow across the balloon, the received information regarding blood flow across the balloon based at least in part on the measured velocity.

Example 141. The method of any example herein, particularly example 140, further comprising, based at least in part on the measured velocity of blood flow, determining a desired inflation rate, the desired inflation rate negatively correlated with the measured velocity of blood flow, wherein the received information regarding blood flow across the balloon based at least in part on the desired inflation rate.

Example 142. The method of any example herein, particularly example 141, wherein the rate of inflation of the balloon is based at least in part on the determined desired inflation rate.

Example 143. The method of any example herein, particularly example 141 or 142, further comprising comparing the measured velocity of blood flow to a plurality of respective predetermined parameters, the desired inflation rate determined based at least in part on an outcome of the comparison to the respective predetermined parameters.

Example 144. The method of any example herein, particularly any one of examples 140 to 143, further comprising, based at least in part on the measured velocity of blood flow, determining a desired inflation time, the desired inflation time period positively correlated with the measured velocity of blood flow, wherein the received information regarding blood flow across the balloon is based at least in part on the determined desired inflation time.

Example 145. The method of any example herein, particularly example 144, further comprising stopping the inflation of the balloon when the determined desired inflation time period ends.

Example 146. The method of any example herein, particularly any one of examples 140 to 142, further comprising: based at least in part on the measured velocity of blood flow, determining a desired inflation time, the desired inflation time period positively correlated with the measured velocity of blood flow, wherein the received information regarding blood flow across the balloon is based at least in part on the determined desired inflation time; and comparing the measured velocity of blood flow to a plurality of respective predetermined parameters, the desired inflation time determined based at least in part on an outcome of the comparison to the respective predetermined parameters.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

What is claimed is:

1. A delivery apparatus comprising:
a balloon movable between a deflated state and an inflated state, the balloon comprising:
an internal cavity;
a central portion disposed around the cavity and extending along a length between a leading portion and a trailing portion of the balloon, wherein the central portion comprises at least two lobes spiraling around a longitudinal axis of the balloon and along the length of the central portion, and at least two depressed portions extending between the lobes; and
a balloon catheter coupled to the balloon, wherein the balloon catheter comprises a balloon catheter lumen in fluid communication with the cavity of the balloon via one or more openings of the balloon catheter;
wherein, when the balloon is in the inflated state within a target lumen, at least two channels are provided between the depressed portions and an inner surface of the target lumen to allow blood to flow through the channels and across the balloon.

2. The delivery apparatus of claim 1, wherein the lobes and the depressed portions together form a cross-sectional contour devoid of acute or right angles between the lobes and the depressed portions.

3. The delivery apparatus of claim 1, wherein the balloon is not wound around any other shaft or catheter extending along the longitudinal axis.

4. The delivery apparatus of claim 1, wherein the delivery apparatus is devoid of constriction elements helically disposed around the balloon.

5. The delivery apparatus of claim 1, wherein the delivery apparatus is devoid of biasing means disposed in the cavity, configured to press against the balloon in the inflated state to force it into a helically-shaped configuration.

6. The delivery apparatus of claim 1, wherein the at least two lobes comprise three lobes, and wherein the at least two depressed portions comprise three depressed portions.

7. The delivery apparatus of claim 1, wherein the leading and the trailing portions taper, in the inflated state, from the respective ends of the central portion to narrower diameters at their opposite ends.

8. The delivery apparatus of claim 1, wherein the leading and the trailing portions have non-circular cross-sectional shapes along at least a portion of tapering segments thereof in the inflated state.

9. The delivery apparatus of claim 1, further comprising at least one flow sensor configured to detect the presence of blood flow across the balloon.

10. The delivery apparatus of claim 9, wherein the at least one flow sensor is attached to the balloon.

11. The delivery apparatus of claim 10, wherein the at least one flow sensor is attached to at least one of the depressed portions.

12. The delivery apparatus of claim 1, further comprising at least one force sensor.

13. The delivery apparatus of claim 12, wherein the at least one force sensor is attached to a lobe outer surface of at least one of the lobes.

14. The delivery apparatus of claim 13, wherein the lobe outer surface defines a width that is at least as great as the width of the force sensor.

15. A method comprising:
delivering a balloon in a deflated state thereof, mounted on a balloon catheter, through the vasculature of a patient to a treatment site, the balloon comprising an internal cavity and a central portion disposed around the cavity;
injecting inflation fluid into the internal cavity of the balloon;
receiving information regarding blood flow across the balloon; and
based at least in part on the received information, stopping the injection of the inflation fluid into the balloon,
wherein the injection of the inflation fluid into the internal cavity of the balloon inflates the balloon within a target lumen such that the central portion comprises at least two lobes spiraling around a longitudinal axis of the balloon and along a length of the central portion, and at least two depressed portions extending between the lobes, and
wherein blood is permitted to pass through at least two channels formed between the depressed portions and the target lumen.

16. The method of claim 15, wherein the injecting the inflation fluid comprises:
injecting a first volume of the inflation fluid into the balloon; and
injecting a second volume of the inflation fluid into the balloon, wherein the second volume of the inflation fluid is determined based at least in part on the received information.

17. The method of claim 16, wherein the first volume of the inflation fluid is a first predetermined fraction of a nominal inflation volume.

18. The method of claim 16, further comprising injecting an additional volume of the inflation fluid into the internal cavity of the balloon until a first predetermined condition is satisfied.

19. The method of claim 18, further comprising measuring an amount of force between the balloon and tissue of the patient, or between the balloon and a prosthetic device, wherein the first predetermined condition is associated with the measured amount of force.

20. The method of claim 15, further comprising measuring the velocity of blood flow across the balloon, the received information regarding blood flow across the balloon based at least in part on the measured velocity.
